(19) United States
(12) Patent Application Publication
(10) Pub. No.: US 2023/0270919 A1
(43) Pub. Date: Aug. 31, 2023

(54) IMPROVED MECHANICAL PROPERTIES OF IMPLANTABLE VASCULAR GRAFTS

(71) Applicant: The Johns Hopkins University, Baltimore, MD (US)

(72) Inventors: Sharon Gerecht, Severna Park, MD (US); Morgan B. Elliott, Baltimore, MD (US); Hai - Quan Mao, Baltimore, MD (US); Jessica Shen, Baltimore, MD (US)

(21) Appl. No.: 17/766,569

(22) PCT Filed: Oct. 15, 2020
(86) PCT No.: PCT/US2020/055662

§ 371 Date: Apr. 5, 2022

Related U.S. Application Data
(60) Provisional application No. 62/915,230, filed on Oct. 15, 2019.

(51) Int. Cl. A61L 27/52; A61L 27/38; A61L 27/50

(52) U.S. Cl. CPC A61L 27/52; A61L 27/3808; A61L 27/507; A61L 2400/12

(57) ABSTRACT

Described are methods of making vascular grafts from man-made tubular scaffolds, tubular scaffolds, and methods implanting vascular grafts comprising tubular scaffolds into subjects. The tubular scaffolds of the present invention are made of hydrogel nanofibers that have internally aligned polymer chains and may be cellularized.

### REFERENCE TO RELATED APPLICATIONS

This application claims the benefit of U.S. Provisional Patent Application No. 62/915,230, filed on Oct. 15, 2019, which is hereby incorporated by reference for all purposes as if fully set forth herein.

### STATEMENT OF GOVERNMENTAL INTEREST

This invention was made with government support under grant nos. CBET1054415 and DMR1410240 awarded by the National Science Foundation. The government has certain rights in the invention.

### BACKGROUND OF THE INVENTION

The development of an in vitro microvascular model is of prominent interest for a wide array of applications, including the study and treatment of cardiovascular disorders, which remains the leading cause of death worldwide, contributing to 30% of mortalities (Mozaffarian D, 2015). Other applications include tissue engineering of vascularized organs, drug testing, blood brain barrier modelling, and even cancer treatment since angiogenesis is a hallmark of tumor growth. Understanding these processes has critical therapeutic and scientific potential.

Several groups have reported success in creating capillary beds as well as large vessels (>6 mm diameter) artificially (Kelm, et al., 2010, L'heureux, et al., 1998, Quint, et al., 2011). However, creating functional microvasculature, the connecting bridge between them, has been elusive as it requires complex interactions between endothelial cells (ECs), perivascular cells (PCs), and the extracellular matrix (ECM) proteins on which they reside. ECs comprise the tunica intima or innermost lining of the vessels, while PCs, namely vascular smooth muscle cells (vSMCs) or pericytes, make up the tunica media or middle layer and provide the contractility necessary for vasoreactivity (Bruce Alberts, 2002). In between these two layers lies a basal lamina composed of several ECM proteins (Carmeliet and Jain, 2000, Jain, 2003). The specific composition as well as the organization and arrangement of both cellular and acellular components are necessary for proper microvasculature development, maturation, stability, and function (Braverman and Yen, 1977, Carmeliet and Jain, 2000, Jain, 2003). Specifically, it has been shown that in small vasculature, EC nuclei and cytoskeleton are aligned in the direction of blood flow; while PCs wrap around the endothelium, providing support and structure to the continuous EC monolayer (Ives, et al., 1986, Williams, 1998). The ECM of each layer has been shown to provide support and relay an array of different biomechanical and biochemical cues to vascular cells (Davis and Senger, 2005), as well as to have either a longitudinal or circumferential orientation depending on its location within the vessel (Schriefl, et al., 2011).

Previously the inventors developed a modified electrospinning technique that could be used to create hydrogel microfibers of different biomaterials with an aligned internal and external nanotopography and enhanced mechanical properties (Zhang, et al., 2014 and U.S. Pat. No. 10,119,202). The inventors demonstrated that fibrin hydrogel microfibers created with this technique could be used to guide the formation of an aligned endothelium by seeding endothelial colony forming cells (ECFCs) on the outer surface, and that these cells deposited extracellular matrix (ECM) proteins, including fibronectin (Fn), collagen IV (Col IV), and laminin (Lmn), to create a subendothelial membrane. The inventors further demonstrated that these proteins were deposited following a circumferential orientation and that this process was regulated by the substrate's curvature (Barreto-Ortiz, et al., 2013).

More recently, the inventors showed the microfiber's 3D geometry also enhanced the quantity of ECM proteins deposited by ECFCs as well as mural cells, namely pericytes and vSMCs, which were found to deposit collagens I, III, and IV, as well as Fn and Lmn. vSMCs were also found to deposit elastin (Eln). Altogether, these proteins made up different layers of the vascular wall and their optimized deposition enabled the generation of self-standing microvascular structures in vitro for the first time. In this model, we optimized a protocol where fibrin microfibers were cultured for 5 days with ECFCs and further co-cultured for 5 more days with vSMCs or pericytes, after which the fibrin core was degraded using plasmin to create a multicellular luminal microvascular structure with an aligned endothelium and a fully invested perivascular cell layer (Barreto-Ortiz, et al., 2015). In order to continue the process of microvascular development, it is critical to apply flow through the luminal space. It is thus necessary to establish a protocol to generate a vascular wall robust enough to withstand flow without leaking or bursting and to engineer a system to perfuse the developing vasculature in a controlled manner.

There is also a significant need for small-diameter tissue engineered vascular grafts (sTEVGs, <6 min in diameter) for the treatment of pediatric congenital cardiovascular defects (CCD), which are present in 1% of live births, including 20,000 infants born annually in the United States. Single ventricle cardiac anomalies are the most severe CCDs and require repeated surgical reconstruction to maximize long-term survival. While artificial grafts made of Goretex®, Dacron®, and polyurethanes are the most common for vascular bypass surgeries that require grafts greater than 6 mm in diameter, synthetic sTEVGs have yet to show clinical effectiveness. In fact, the use of synthetic vascular grafts in these procedures remains the leading source of morbidity and mortality due to complications arising from the inability of synthetic grafts to grow as the child develops. While several efforts have been successful in developing sTEVGs (<6 mm), a functional graft has remained elusive due to post-implantation challenges, including thrombogenicity, decreased elasticity, decreased compliance, aneurysmal failure, calcification, and intimal hyperplasia.

The standard treatment for coronary artery disease (CAD) and peripheral artery disease (PAD), which afflict small-diameter arteries, is the use of autologous tissue as a bypass graft. Autologous tissue grafts provide superior outcomes in comparison with synthetic grafts, but lack of vascular tissue in these patients limits autologous tissue reconstruction. Autografts have several disadvantages, including the inconvenience of harvesting and preparing the tissue graft. Design of a sTEVG that matches the native vessel size and mechanical properties; is capable of growing with the patient and incorporating into the patient's vascular tissue; has low thrombogenicity; and exhibits a clinically relevant shelf-life would provide a substantial benefit to pediatric CCD, CAD, and PAD applications by improving patient morbidity and reducing long-term costs.

### SUMMARY OF THE INVENTION

In accordance with a first embodiment, the present invention provides a non-cellularized vascular graft comprising: a tubular scaffold including a hollow core surrounded by one or more sheets comprising dehydrated hydrogel nanofibers with internal polymer alignment.

In accordance with a second embodiment, the present invention provides a cellularized vascular graft comprising a tubular scaffold including a hollow core surrounded by one or more sheets comprising hydrated hydrogel nanofibers with internal polymer alignment; and one or more cell layers attached to the tubular scaffold.

In accordance with a third embodiment, the present invention provides a method of using a vascular graft to treat vascular damage comprising the steps of administering a vascular graft of the present invention (including a non-cellularized and a cellularized vascular graft) to a subject with vascular damage; and treating the vascular damage of the subject.

In accordance with a fourth embodiment, the present invention provides a mesh comprising sheets comprising dehydrated or hydrated hydrogel nanofibers having internally aligned polymer chains wherein each sheet has a controlled nanofiber orientation that is longitudinal, perpendicular, or otherwise angled.

In accordance with a fifth embodiment, the present invention provides a bioreactor comprising: two interior walls forming a right, central, and left chamber; the central chamber comprising a solid tubular scaffold tethered to the two interior walls; a top and a bottom plate in contact with the two interior walls; and the right, the central, and the left chamber each comprises one or more ports to allow perfusion.

In accordance with a sixth embodiment, the present invention provides a method of making a microvascular structure comprising: a bioreactor of the present invention or other culture device; first seeding cells into the center chamber on day 0; second seeding of cells to allow a confluent cell layer to form around a solid hydrogel microfiber made of nanofibers; and culturing to form a microvascular structure comprising the solid microfiber.

In accordance with a seventh embodiment, the present invention provides a perfusion bioreactor comprising: a bioreactor wall forming an enclosure; a port for chamber media changes; a conduit for perfusion traversing the bioreactor wall; and a tubular scaffold, wherein the tubular scaffold is attached to the one or more conduits.

In accordance with a eighth embodiment, the present invention provides a method of making a vascular graft structure with a hollow core such as a sTEVG, as an example, comprising: providing a bioreactor of the present invention or other culture device, preferably a perfusion bioreactor; first seeding cells into the perfusion bioreactor on day 0; potential second or more seeding of cells to allow a confluent cell layer to form on a tubular scaffold of hydrogel nanofibers; culturing to form a vascular structure of cells having a hollow core.

In accordance with a ninth embodiment, the present invention provides a microvascular structure containing a cell wall, made from a solid microfiber comprising a bundle of hydrogel nanofibers having internal alignment of a polymer. As described in the specification, nanofibers are made from a hydrogel polymer such as fibrin, alginate, gelatin, hyaluronic acid, collagen, chitosan, or a combination thereof.

In accordance with a tenth embodiment, the present invention provides a man-made vascular graft such as sTEVG, for example, comprising a tubular scaffold of the present invention (described in greater detail in the specification), and at least one layer of cells on the tubular scaffold.

In accordance with a eleventh embodiment, the present invention provides a method of making a tubular scaffold comprising: electrospinning a hydrogel polymer solution from a biopolymer jet into a thrombin or other type of collection solution that is stationary or moving; rastering the landing position of the biopolymer jet back-and-forth across the collection solution to make a biopolymer sheet of hydrogel nanofibers having an internal alignment of polymer chains; rolling the biopolymer sheet around a PTFE-coated mandrel in any direction such as perpendicular, parallel, or a mixture thereof forming a wall with a thickness; forming a tubular scaffold comprising a hollow core and one or more sheets comprising hydrogel nanofibers with internal alignment of polymer chains and the tubular scaffold has circumferential, longitudinal, or mixed topography; crosslinking the hollow tubular scaffold by chemical or physical methods; dehydrating the hollow tubular scaffold via lyophilization or graded ethanol treatments; removing the dehydrated hollow tubular scaffold from the mandrel; and forming a dehydrated hollow tubular scaffold.

### BRIEF DESCRIPTION OF THE DRAWINGS

FIG. 1 illustrates a schematic of microvascular development process in three-chamber bioreactor using solid microfibers. Bioreactor is divided into three compartments by two Polydimethylsiloxane (PDMS) walls. Longitudinally aligned fibrin microfibers as described in U.S. Pat. No. 10,119,202 and hereby incorporated by reference herein, are tethered in the central compartment in between the two walls. ECFCs are seeded in this compartment and cultured for 5 days, after which SMCs are seeded on top and cultured for 10-15 more days. The core is then degraded using a plasmin solution. Not drawn to scale.

FIG. 2A-2E illustrates a three-chamber bioreactor design. (2A) Design schematic and specifications of top and bottom plates of the bioreactor. Top view of (2B) complete assembled bioreactor and (2C) bottom plate only showing imaging window. (2D) Assembled bioreactor with glass slide and PDMS walls dividing the chamber into three separate compartments filled with PBS (left and center) or DMEM (right). Seeding set-up on (2E) 5 fibrin microfibers with a cell suspension in the center compartment and PBS in the outer compartments.

FIG. 3A-3F illustrates microvascular structures. Structures were developed in three-chamber bioreactor on fibrin microfibers with ECFCs for 5 days followed by culture with SMCs for 10-15 days before treatment with fibrin degradation media. (3A) Epifluorescence images of whole microvascular structure. Scale=500 μm. Confocal projections of structures at (3B) low (scale=200 μm) (3C) mid (scale=100 μm) and (3D) high magnification (scale=20 μm). (3E) Orthogonal projections of structures at (I) luminal space, (II) inner cell layer, (III) mid cell layer, and (IV) outer cell layer. Scale=50 μm. (3F) High magnification orthogonal projections of structures at (I) mid cell layer and (II) outer cell layer. Scale=20 μm. (3G) Epifluorescence images of cross-sectional cryo-sections of structures with incomplete degradation. Scale=50 μm. Elastin (Eln, red), Col IV (magenta), F-actin (green), and nuclei (blue). Double-headed arrow shows microfiber longitudinal direction.

FIG. 4A-4F illustrates fabrication and properties of fibrin microfibers and tubular scaffolds. Workflow begins with (4A) fibrin hydrogel microfibers being spun into a sheet by rastering the landing position of the biopolymer jet on a rotating collection solution. (4B) Sheets are collected by placing a PTFE-coated mandrel on the resultant hydrogel sheet either perpendicular or parallel to the fiber orientation. (4C) Sheets are then (i) wrapped, and (ii) the PTFE-coated mandrel is removed after dehydration, (iii) yielding hollow fibrin tubes with circumferential (Left) or longitudinal (Right) alignment. (4D) Representative SEM micrographs of (i) circumferentially aligned hollow tube and (ii) a stretch-dried, longitudinally aligned fibrin fiber bundle. Quantification of (iii) longitudinally aligned fibrin microfiber orientation and (iv) orientation distribution for grafts dehydrated via graded ethanol (EtOH) and lyophilization (Lyo) treatments relative to the longitudinal graft axis (0°). (Scale bars: i, 200 μm; ii, 40 μm) (4E) Representative longitudinal and cross-section SEM micrographs of (i and ii) Lyo-treated and (iii and iv) EtOH-treated longitudinally aligned hollow tubes. (Scale bar: 400 μm) (4F) Mechanical properties, including (i) elastic modulus, (ii) axial STF, (iii) axial UTS, (iv) toughness, and (v) outer diameter of fibrin tubes dried using the Lyo and EtOH treatments (n=3-5). *p<0.05; ****p<0.0001.

FIG. 5A-5D illustrates a single chamber bioreactor design and microvascular development process on fibrin tubes. (5A) Longitudinally aligned fibrin tubes are tethered on the 25Ga needles between the two PDMS walls within the glass chamber. ECFCs are seeded in this compartment and cultured for 5-7 days, after which SMCs are seeded on top and cultured for 5-7 more days. Resulting structures are then perfused using a peristaltic pump. (5B) Side view design schematic and specifications of single chamber bioreactor. Not drawn to scale. (5C) Top view of completely assembled bioreactor filled with PBS (top) and a cell suspension (bottom). (5D) Two parallel perfusion set-ups powered by a peristaltic pump, featuring a media reservoir, air filter, and two single chamber bioreactor chambers.

FIG. 6A-6H illustrates microvascular structures developed in single chamber bioreactor. Structures at day 7 of ECFC culture (n=3) presented as (6A) maximum intensity and (6B) 3D cross-sectional reconstruction at low magnification (scale=200 μm); (6C) maximum intensity projections at mid magnification (scale=100 μm); and (6D) 3D cross-sectional reconstruction at high magnification. Structures after 7 days of ECFC culture followed by 7 days of SMC co-culture (n=2) presented as (6E) stitched maximum intensity projection at low magnification (scale=200 μm); (6F) maximum intensity projection at mid magnification (scale=100 μm); (6G) Orthogonal projections at (I) inner and (II) outer cell layers showing ECFC and SMC layers, respectively (scale=50 μm); and (6H) 3D cross-sectional reconstruction at mid magnification. All 3D reconstructions depict a luminal cross-sectional view of the microvascular tube structures at a slight angle to enable visualization of the lumen and outer surface. Double-headed arrow shows microfiber longitudinal direction. Yellow arrowheads indicate cells underneath outer cell layer.

FIG. 7A-7C illustrates perfusion through engineered microvasculature. Hollow fibrin microfibers cultured with ECFCs for 5 days followed by co-culture with vSMCs for 5 more days and further cultured under static conditions or with luminal perfusion for 3 days (7A) Manual perfusion. Arrow points to leading edge of medium during flow. (7B) Confocal projections of ECM in (I) static control (II) 3 day perfusion. Scale bars=200 μm. (7C) Measurement of lumen diameter. Error bars represent SEM. Significance levels in the distribution represented by ***p<0.001.

FIG. 8 illustrates tubular scaffolds of the present invention used to create in-vitro vascular structures such as grafts having a hollow core. These tubular scaffolds have been dried to extend shelf life and for shipping.

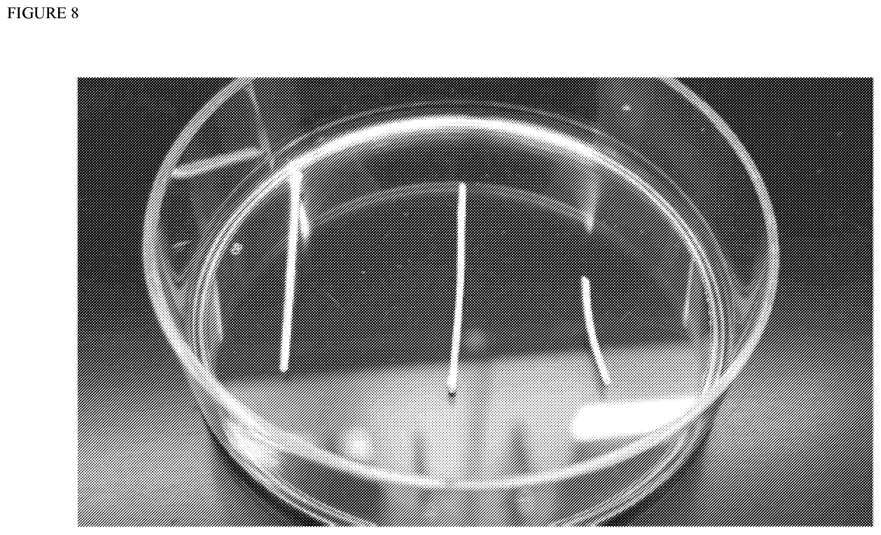

FIG. 9. Fiber Dehydration Process for Storage of Hollow Fibrin Tubular scaffolds. The number of layers of fibrin used to wrap the mandrel determines the wall thickness of the hollow tubular scaffolds, which can be easily and precisely altered. Pre-dehydration hollow fibrin tubular scaffolds that are left on the mandrels and in DI water (left). During the graded ethanol dehydration, the hollow tubular scaffolds are left on the mandrel and placed in increasing concentrations of ethanol (center). Finally, once the hollow tubular scaffolds are completely dehydrated, the mandrels are removed, and the self-standing tubular scaffolds can be placed in storage or transported (right).

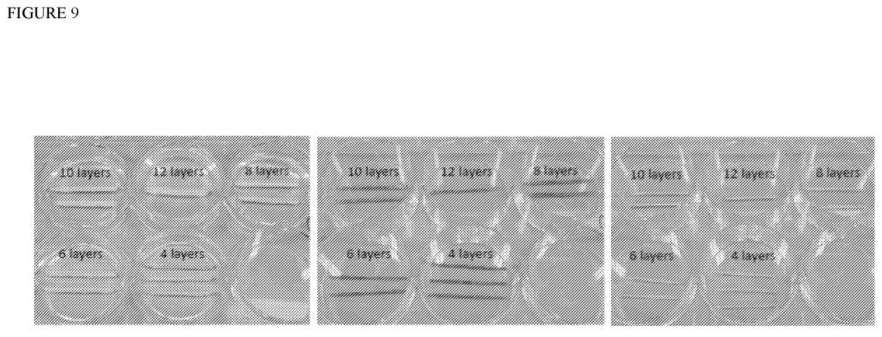

FIG. 10. Internal cellularization of hollow fibrin tubular scaffolds. Samples at day 3-4 of ECFC culture (n=3) presented as maximum intensity confocal projections at, (10A) low (scale=200 μm), (10B) mid (scale=200 μm), peak magnifications (10Ci, scale=20 μm) (10Cii, scale=50 μm), and (10D) orthogonal projection (scale=50 μm). F-actin (green), nuclei (blue), lumen (L), and sTEVG wall (W). Double-headed arrows show microfiber longitudinal direction.

FIG. 11. Implantation of Acellular Hollow Fibrin Tubular scaffolds in Murine Model. An image of the fibrin tubular scaffold (double headed arrow) implanted as an abdominal aorta (Ao) interposition graft in a murine model. The inferior vena cava (IVC) is indicated. The graft was anastomosed to the native abdominal aorta (single headed arrows) and blood flow was observed after the clamps were removed. Pulsation was visible in the graft and artery, which was indicative of arterial flow.

FIG. 12. sTEVGs at 8 weeks post-implantation. Staining for the various markers indicated (arrowheads indicate elastin fibrils or fibers). Lumen (L), Sleeve (S), and fibrin sTEVG (F) (scale=30 μm, n=4).

FIG. 13. Storage of Fibrin Microfiber Tubes.

FIG. 14. Illustrates the range of fibrin tube inner diameter that can be achieved. The left shows the relative scale of fibrin tubes with 0.60 mm and 5.00 mm inner diameters. The right are SEM images showing the ability to maintain controlled nanofiber alignment within the larger 5.00 mm inner diameter structures.

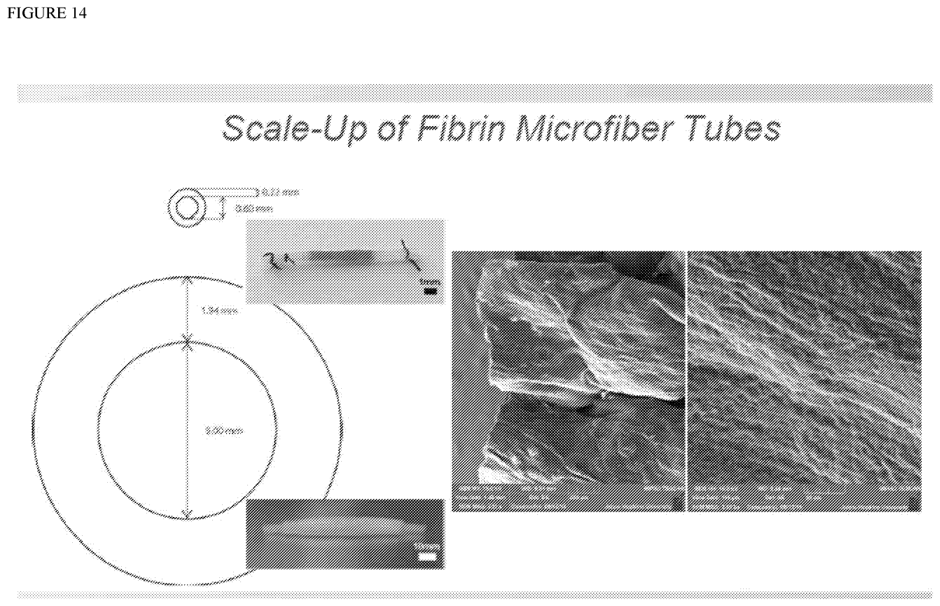

FIG. 15. Mechanical properties of various graft configurations and controls. Graft configuration diagrams indicate the combinations of longitudinally (black) and circumferentially (gray) wrapped microfiber sheets around the lumen (L) as well as the PCL sheath (green; not to scale). Human vessel values provided as reference for large animal model relevance (S. Pashneh-Tala, S. MacNeil, F. Claeyssens, The tissue-engineered vascular graft-past, present, and future. Tissue Eng. Part B Rev. 22, 68-100 (2016). 30. J. Johnson, D. Ohst, T. Groehl, S. Hetterscheidt, M. Jones, Development of novel, bi-oresorbable, small-diameter electrospun vascular grafts. J. Tissue Sci. Eng. 6, 1000151 (2015); R. Marx et al., Morphological differences of the internal thoracic artery in patients with and without coronary artery disease—Evaluation by duplex-scanning. Eur. J. Cardiothorac. Surg. 20, 755-759 (2001); Z. Teng, D. Tang, J. Zheng, P. K. Woodard, A. H. Hoffman, An experimental study on the ultimate strength of the adventitia and media of human atherosclerotic carotid arteries in circumferential and axial directions. J. Biomech. 42, 2535-2539 (2009); B. A. Hamedani, M. Navidbakhsh, H. A. Tafti, Comparison between mechanical properties of human saphenous vein and umbilical vein. Biomed. Eng. Online 11, 59 (2012).) Not applicable (N/A): Burst pressure could not be collected for the native mouse abdominal aorta, as it had too many branches to be stably pressurized during pressure myography; death after implantation occurred significantly later for the multidirectional graft group (n=3-6). *p<0.05; **p<0.01; ****p<0.0001.

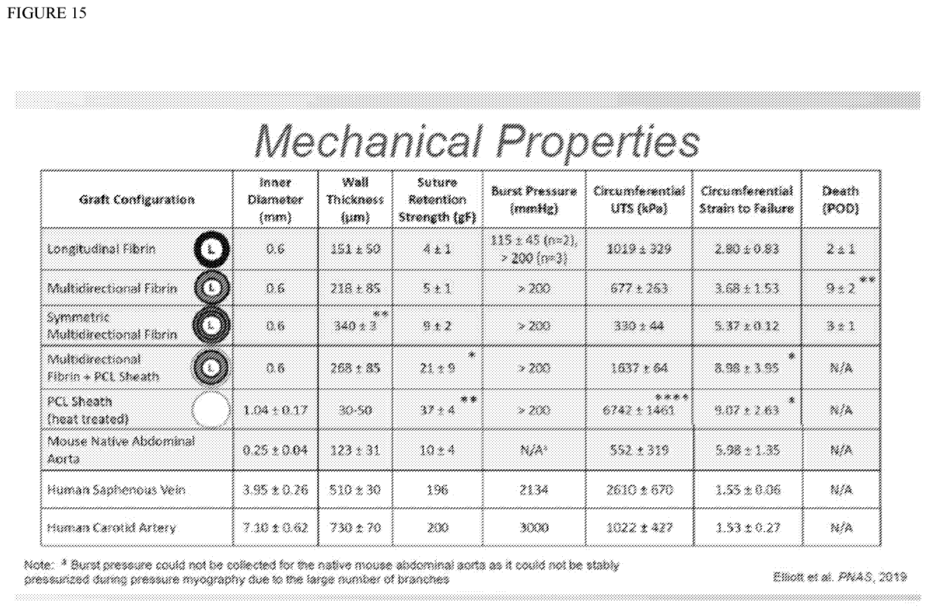

FIGS. 16A-16C. Crosslinking of fibrin hydrogel microfibers. A workflow schematic for fabrication of fibrin hydrogel microfibers involving electrospinning and crosslinking. (16A) Fibrinogen is first enzymatically crosslinked with thrombin to cleave fibrinopeptides A and B, allowing the D and E globular domains to interact and form a fibrin polymer during the electrospinning process. Electromechanical stretching and rastering the landing position of the biopolymer jet allows formation of an aligned fibrin microfiber sheet. After rolling the sheets to create a hollow fibrin microfiber tube, (16B) the fibrin hydrogel D globular domains are chemically crosslinked with EDC/NHS treatment to form D-dimers. Lastly, the microfiber tube is (16C) physically crosslinked and hydrogen bonds are formed with graded ethanol dehydration. This results in compaction of the fibrin microfibers. The microfiber tube can now be stored and rehydrated for future use.

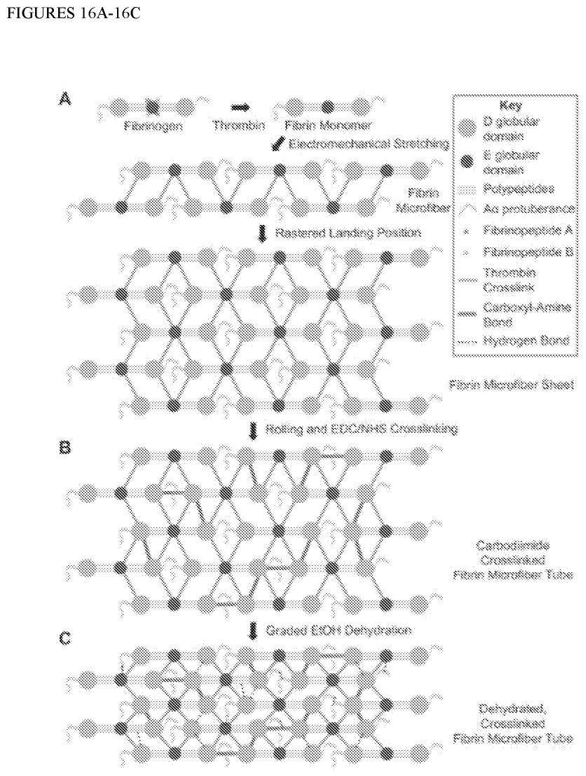

FIGS. 17A-17F: Mechanical Properties of Stored sdVGs. 17A. Circumferential ultimate tensile stress (UTS), 17B. Circumferential strain to failure (STF), 17C. Young's modulus, 17D. Modulus of toughness, 17E. Modulus of resilience, and 17F. Plasticity of sdVGs that underwent accelerated aging. Values are reported as mean±standard deviation. Two-way ANOVA with Tukey's multiple comparison test was used to determine significance (n=8-11, *p<0.05, **p<0.01, ***p<0.001, and ****p<0.0001). Black stars indicate significance over storage time. Yellow stars indicate significance between ambient temperature storage groups.

FIGS. 18A-18B: Rehydration time of sdVGs. Rehydration time has no significant effect on 18A. Circumferential ultimate tensile stress (UTS) or 18B. Circumferential strain to failure (STF). Therefore, time of use after rehydration is not critical. Time 0 is when we began rehydration, which is finished by 0.5 hrs. (n=6)

FIG. 19: Swelling ratio of stored sdVGs. There is no significant difference in swelling ratio over storage time or between different storage conditions for grafts that have been stored in real time. Storage of grafts does not affect the swelling ratio of our electrospun hydrogel structure (n=4-9).

FIGS. 20A-20D: Fabrication of Hollow Fibrin Hydrogel Microfiber sdVGs. 20A. Fibrinogen is electrospun to form aligned sheets of fibrin microfibers. 20B. A teflon-coated mandrel is placed perpendicular (left) or parallel (right) to the hydrogel fiber orientation in preparation to be rolled circumferentially or longitudinally, respectively. The inset is a newly electrospun aligned fibrin sheet with controlled microtopography. 20C. The mandrel is rolled longitudinally, circumferentially, and longitudinally to collect the fibrin sheet. 20D. After being crosslinked and dehydrated, the hollow fibrin microfiber tubes are then slid off the mandrels and placed into the appropriate storage environment. The grafts are rehydrated before testing.

FIGS. 21A-21F: Mechanical properties of sdVGs stored in real time. 21A. Circumferential ultimate tensile stress (UTS), 21B. Circumferential strain to failure (STF), 21C. Young's modulus, 21D. Modulus of toughness, 21E. Modulus of resilience, and 21F. Plasticity of sdVGs that underwent real time storage. Controls were tested within 5 days of fabrication. Values are reported as mean±standard deviation. Two-way ANOVA with Tukey's multiple comparison test was used to determine significance (n=7-11, *p<0.05, **p<0.01, ****p<0.0001, and N.S. is no significance). Black stars indicate significance over storage time. Yellow stars indicate significance between ambient temperature storage groups.

FIGS. 22A-22F: Comparison of accelerated aging and real time storage of sdVGs. 22A. Circumferential UTS, 22B. Circumferential STF, 22C. Young's modulus, 22D. Modulus of toughness, 22E. Modulus of resilience, and 22F. Plasticity of sdVGs that underwent accelerated aging (accel) or real time (real) storage. Values are reported as mean±standard deviation. Two-way ANOVA with Sidak's multiple comparison test was used to determine significance (n=7-9, *p<0.05, **p<0.01, and ****p<0.0001). Black stars indicate significance between accelerated and real time groups.

### DETAILED DESCRIPTION OF THE INVENTION

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “biopolymer jet” is meant the thin stream of biopolymer fluid attained by application of an electric field to the needle tip, that is then collected onto a surface or into a bath, which may be stationary or moving.

By “drip seeding” is meant the act of placing cells onto a surface, like a graft or the tubular scaffold, by pipetting a concentrated cell solution onto the material surface. In other words, the cell solution is “dripped” onto the material surface. This allows cells to be placed in specific locations or localized areas for celluarization of the surface. The cells are allowed to adhere to the surface before other manipulations or procedures are performed.

By “effective amount” is meant the amount of a required substance, such as a microvascular structure or graft, to ameliorate the symptoms of a disease relative to an untreated patient. The effective amount (or length) of graft(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

By “gravitational seeding” or “bulk gravitational seeding” or “rotational based seeding” is meant the act of placing cells onto a structure, like a graft or the tubular scaffold, by pipetting a concentrated cell solution into a chamber filled with fluid, in which the structure is suspended. The chamber is then rotated to allow the cells to remain suspended in the fluid and come into contact with the structure, to which they can adhere. This allows the entire structure to be covered for cellularization in a “bulk” manner. The cells are allowed to adhere to the surface before other manipulations or procedures are performed.

By “internal rotational based seeding” is meant the act of placing cells into a structure, like a graft or the hollow tubular scaffold, by pipetting a concentrated cell solution into the hollow lumen and filling the lumen with fluid. The structure is then rotated to allow the cells to remain suspended in the fluid and come into contact with the internal surface of the structure, to which they can adhere. The cells are allowed to adhere to the surface before other manipulations or procedures are performed, including the removal of the cell solution and replacement with fresh fluid.

By “microfiber” is meant a solid tubular structure made up of a bundle of nanofibers.

By “perfusion-based seeding” or “internal, perfusion-based seeding” is meant the act of placing cells onto a structure, like a graft or the tubular scaffold, by flowing a cell solution through or around the structure. To internally seed a hollow structure, the cell solution is perfused through the hollow lumen. The cells are allowed to adhere to the surface before other manipulations or procedures are performed, including the removal of the cell solution and replacement with fresh fluid.

A “reference” refers to a standard or control conditions such as a sample (human cells) or a subject that is free, or substantially free, of a composition of method of the present invention. For example, a reference subject having a vascular injury without a vascular structure of the present invention implanted. Such a reference subject may be compared with a subject having a vascular injury with a microvascular structure of the present invention implanted. The subject could be the same.

A “tubular scaffold” generally means a structure comprising a sheet of hydrogel nanofibers forming a circumference around a hollow core. Many embodiments of tubular scaffolds and their uses are provided.

By “vascular graft” is meant a man-made acellular or cellular tubular scaffold of the present invention that may include a cellular structure. The vascular grafts of the present invention may be used to treat vascular disease, as an example. A small-diameter tissue engineered vascular graft (sTEVG) is a vascular graft having a diameter <6 mm. The vascular graft may taper or vary in size to match the existing vasculature and subject needs.

As used herein, the term “subject” is intended to refer to any individual or patient to which the method described herein is performed. Generally, the subject is human, although as will be appreciated by those in the art, the subject may be an animal. Thus other animals, including mammals such as rodents (including mice, rats, hamsters, and guinea pigs), cats, dogs, rabbits, farm animals including cows, horses, goats, sheep, pigs, etc., and primates (including monkeys, chimpanzees, orangutans, and gorillas) are included within the definition of subject.

By “tumble” is meant the rotation of a structure or device around a fixed axis so that the cells in solution are kept suspended. This technique is part of the gravitational based seeding, bulk gravitational based seeding, internal rotational based seeding, and rotational based seeding methods.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

Any compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

In accordance with a first embodiment, the present invention provides a non-cellularized vascular graft comprising: a tubular scaffold including a hollow core surrounded by one or more sheets comprising dehydrated hydrogel nanofibers with internal polymer alignment. Vascular grafts of the present invention (including both a non-cellularized and/or a cellularized vascular graft) may be made of sheets wherein each sheet has the same or different alignment of nanofibers. Examples include sheets comprising longitudinally aligned dehydrated hydrogel nanofibers, sheets comprising circumferentially aligned dehydrated hydrogel nanofibers, sheets having other alignments of dehydrated hydrogel nanofibers relative to the longitudinal axis of the tubular scaffold, and sheets having no alignment of nanofibers. Suitable vascular grafts of the present invention, i.e. non-cellularized and cellularized grafts, have a hollow core with an inner diameter in the range of 0.1 mm to 6 mm and the one or more sheets may have a combined thickness in the range of 5 nm to 3000 μm, 4 nm to 3000 μm, 3 nm to 3000 μm, 1 nm to 3000 μm, or 0.5 nm to 3000 μm, as examples. A non-cellularized vascular graft of the present invention may have an average circumferential Ultimate Tensile Stress (UTS) in a range of 40 kPa to 600 kPa, 50 kPA to 500 kPa, 60 kPa to 400 kPA, 70 kPa to 300 kPa, or 80 kPa to 200 kPa. A non-cellularized vascular graft of the present invention may have and an average circumferential Strain to Failure (STF) in a range of 1 to 5, 1.5 to 4.5, 2.0 to 4.0, or 2.5 to 3.5. A non-cellularized vascular graft of the present invention may have an elastic modulus in the range of 20 kPa to 300 kPa, 30 kPa to 250 kPa, 40 kPa to 200 kPa, 50 kPa to 150 kPa, or 60 kPa to 100 kPa. A non-cellularized vascular graft of the present invention having a toughness in the range of 40 kPa to 1000 kPa, 50 kPa to 900 kPa, 60 kPa to 800 kPa, 70 kPa to 700 kPa, 80 kPa to 600 kPa, 90 kPa to 500 kPa, 100 kPa to 400 kPa, or 200 kPa to 300 kPa.

In accordance with a second embodiment, the present invention provides a cellularized vascular graft comprising a tubular scaffold including a hollow core surrounded by one or more sheets comprising hydrated hydrogel nanofibers with internal polymer alignment; and one or more cell layers attached to the tubular scaffold. The one or more cell layers may be composed of ECs, vSMCs, PCs, or a combination thereof that may be attached internally, externally, or a combination thereof to the tubular scaffold, and may have a thickness corresponding to a particular application. The one or more cell layers may have a combined thickness in the range of 10 μm to 300 μm, for example. Because of the structural characteristics of tubular scaffolds having nanofibers with internal polymer alignment, cells, such as endothelial colony forming cells, align in the direction of flow on the tubular scaffold. A cellularized vascular graft of the present invention may have an average circumferential Ultimate Tensile Stress (UTS) in a range of 40 kPa to 600 kPa, 50 kPA to 500 kPa, 60 kPa to 400 kPA, 70 kPa to 300 kPa, or 80 kPa to 200 kPa. A cellularized vascular graft of the present invention may have and an average circumferential Strain to Failure (STF) in a range of 1 to 5, 1.5 to 4.5, 2.0 to 4.0, or 2.5 to 3.5. A cellularized vascular graft of the present invention may have an elastic modulus in the range of 20 kPa to 300 kPa, 30 kPa to 250 kPa, 40 kPa to 200 kPa, 50 kPa to 150 kPa, or 60 kPa to 100 kPa. A cellularized vascular graft of the present invention having a toughness in the range of 40 kPa to 1000 kPa, 50 kPa to 900 kPa, 60 kPa to 800 kPa, 70 kPa to 700 kPa, 80 kPa to 600 kPa, 90 kPa to 500 kPa, 100 kPa to 400 kPa, or 200 kPa to 300 kPa.

In accordance with a third embodiment, the present invention provides a method of using a vascular graft to treat vascular damage comprising the steps of administering a vascular graft of the present invention (including a non-cellularized and a cellularized vascular graft) to a subject with vascular damage; and treating the vascular damage of the subject when compared to a reference subject who has not been administered a vascular graft. A vascular graft may be administered by any suitable means including vascular bypass surgery. Vascular damage may occur to a vascular structure, such as an artery, as an example. The vascular grafts of the present invention may be implanted within the damage area as a means of treating the damage. Vascular damage may be caused by trauma or vascular disease such as congenital cardiovascular defect (CCD), coronary artery disease (CAD), or peripheral artery disease (PAD), as examples.

In accordance with a fourth embodiment, the present invention provides a mesh comprising sheets comprising dehydrated or hydrated hydrogel nanofibers having internally aligned polymer chains wherein each sheet has a controlled nanofiber orientation that is longitudinal, perpendicular, or otherwise angled. A mesh of the present invention may have an average circumferential Ultimate Tensile Stress (UTS) in a range of 40 kPa to 600 kPa, 50 kPA to 500 kPa, 60 kPa to 400 kPA, 70 kPa to 300 kPa, or 80 kPa to 200 kPa. A mesh of the present invention may have and an average circumferential Strain to Failure (STF) in a range of 1 to 5, 1.5 to 4.5, 2.0 to 4.0, or 2.5 to 3.5. A mesh of the present invention may have an elastic modulus in the range of 20 kPa to 300 kPa, 30 kPa to 250 kPa, 40 kPa to 200 kPa, 50 kPa to 150 kPa, or 60 kPa to 100 kPa. A mesh of the present invention may have a toughness in the range of 40 kPa to 1000 kPa, 50 kPa to 900 kPa, 60 kPa to 800 kPa, 70 kPa to 700 kPa, 80 kPa to 600 kPa, 90 kPa to 500 kPa, 100 kPa to 400 kPa, or 200 kPa to 300 kPa.

In accordance with a fifth embodiment, the present invention provides a bioreactor comprising: two interior walls forming a right, central, and left chamber; the central chamber comprising a solid tubular scaffold tethered to the two interior walls; a top and a bottom plate in contact with the two interior walls; and the right, the central, and the left chamber each comprises one or more ports to allow perfusion. In some embodiments, a bioreactor will contain one or more solid hydrogel microfibers that has a longitudinally aligned nanotopography comprising biodegradable, electrostretched hydrogel polymer fibers with internal alignment and having more than one nanofiber. The nanofibers used in the present invention may be made of any suitable material such as fibrin, alginate, gelatin, hyaluronic acid, collagen, chitosan, or a combination thereof. The microfibers may be longitudinally aligned. The walls of a bioreactor may comprise a polymer selected from the group consisting of polydimethylsiloxane (PDMS), hydrogel, plastic, glass, or a combination thereof; and may also comprise an imaging window enabling live imaging. The top and bottom plates of a bioreactor may be sealed by any suitable means such as by a vacuum grease, for example.

In accordance with a sixth embodiment, the present invention provides a method of making a microvascular structure comprising: a bioreactor of the present invention or other culture device; first seeding cells into the center chamber on day 0; second seeding of cells to allow a confluent cell layer to form around a solid hydrogel microfiber made of nanofibers; and culturing to form a microvascular structure comprising the solid microfiber. In the methods of the present invention, the cells may be tumbled when cultured and the second seeding is cultured for at least 6 days from day 0, for example. A third seeding may occur within 10 to 15 days of day 0, for example. A fourth, fifth, or six or more seedings may occur depending upon the particular application of microvascular structure created by the method. Each seeding may be of the same or of a different cell type. In addition, the more seedings performed in a method of the present invention increases the diameter and wall thickness of a microvascular structure being produced. Overall, culturing may continue for up to 30 days from day 0 to form a microvascular structure of the present invention, for example. Any suitable method of seeding may be used such as drip seeding, bulk gravitational seeding, perfusion-based seeding, rotational based seeding, or a combination thereof. Suitable cells used in the present invention include vascular cells, endothelial colony forming cells (ECFCs), perivascular cells (PCs), endothelial cells (ECs), vascular smooth muscle cells, pluripotent stem cells, pluripotent stem cell derived vascular cells, fibroblasts, or a combination thereof. A microvascular structure comprising a solid tubular scaffold core may be treated with plasmin to degrade the solid hydrogel microfiber forming a microvascular structure with a hollow core. Then liquid may flow through the ports of the bioreactor into the microvascular structure having a hollow core.

In accordance with a seventh embodiment, the present invention provides a perfusion bioreactor comprising: a bioreactor wall forming an enclosure; a port for chamber media changes; a conduit for perfusion traversing the bioreactor wall; and a tubular scaffold, wherein the tubular scaffold is attached to the one or more conduits. Any conduit for perfusion may be used that enables the flow of liquid through the tubular scaffold. Such liquids include media, blood, plasma, phosphate buffer saline (PBS), or a combination thereof, as examples. The tubular scaffolds used in the present invention are made of hydrogel nanofibers and may include a polymer selected from the group consisting of fibrin, alginate, gelatin, hyaluronic acid, collagen, chitosan, or a combination thereof, for example. A tubular scaffold of the present invention may have a diameter of the hollow core in the range of 100 μm to 6 mm, 200 μm to 5 mm, 300 μm to 4.5 mm, 400 μm to 4.0 mm, 500 μm to 3.5 mm, 600 μm to 3.0 mm, 700 μm to 2.5 mm, 800 μm to 2.0 mm, or 900 μm to 1.0 mm, for example. In addition, a perfusion bioreactor may include conduits that are needles having a gauge in the range of 34 to 6, 30 to 8, 25 to 10, or 20 to 15, for example.

In accordance with an eighth embodiment, the present invention provides a method of making a vascular graft structure with a hollow core such as a sTEVG, as an example, comprising: providing a bioreactor of the present invention or other culture device, preferably a perfusion bioreactor; first seeding cells into the perfusion bioreactor on day 0; potential second or more seeding of cells to allow a confluent cell layer to form on a tubular scaffold of hydrogel nanofibers; culturing to form a vascular structure of cells having a hollow core.

In accordance with a ninth embodiment, the present invention provides a microvascular structure containing a cell wall, made from a solid microfiber comprising a bundle of hydrogel nanofibers having internal alignment of a polymer. As described in the specification, nanofibers are made from a hydrogel polymer such as fibrin, alginate, gelatin, hyaluronic acid, collagen, chitosan, or a combination thereof, as examples. Microfibers used in the present invention may comprise a diameter in the range of 10 μm-900 μm, 50 μm-800 μm, 100 μm-700 μm, 150 μm-600 μm, 200 μm-500 μm, as examples. A microvascular structure containing a cell wall, surrounding a hollow core maybe created by digesting the microfiber using one or more enzymes. The microvascular structure, or cell wall, will surround a hollow core having a diameter less than or equal to the diameter of the solid microfiber prior to enzyme digestion. For example, the hollow core may have a diameter in the range of 1-900 μm, 1-500 μm, 1-400 μm, 1-300 μm, 1-200 μm, or 1-100 μm for example.

In accordance with a tenth embodiment, the present invention provides a man-made vascular graft such as sTEVG, for example, comprising a tubular scaffold of the present invention (described in greater detail in the specification), and at least one layer of cells on the tubular scaffold. Suitable cells include vascular cells, endothelial colony forming cells (ECFCs), perivascular cells (PCs), endothelial cells (ECs), vascular smooth muscle cells, pluripotent stem cells, pluripotent stem cell derived vascular cells, fibroblasts, or a combination thereof. One embodiment of microvascular structures of the present invention includes at least two distinct cell layers comprising an inner layer adjacent to the tubular scaffold and an outer layer(s) adjacent to the inner layer wherein only the outer layer(s) express smooth muscle protein 22 (SM22) and elastin. In some embodiments of the present invention, microvascular structures may express mature endothelial markers selected from the group comprising von Willebrand factor (vWF), endothelial cell marker cluster of differentiation 31 (CD31), vascular endothelial cadherin (VECad), or a combination thereof. In other embodiments, the microvascular structure may include a deposition of extracellular matrix (ECM) proteins including Collagen IV (Col IV), laminin (Lmn), fibronectin (Fn) or a combination thereof by the cells. Some microvascular structures comprise a deposition of extracellular matrix (ECM) proteins selected from the group comprising Collagen I (Col I), Collagen III (Col III), Collagen IV (Col IV), laminin (Lmn), Elastin, fibronectin (Fn), or a combination thereof by the smooth muscle cells (SMCs). Some microvascular structures express vascular smooth muscle cell (vSMC) markers selected from the group comprising smooth muscle protein 22 (SM22), smoothelin, and smooth muscle myosin heavy chain (SMMHC); and endothelial cell markers selected from cluster of differentiation 31 (CD31), von Willebrand factor (vWF), and vascular endothelial cadherin (VECad).

In accordance with an eleventh embodiment, the present invention provides a method of making a tubular scaffold comprising: electrospinning a hydrogel polymer solution from a biopolymer jet into a thrombin or other type of collection solution that is stationary or moving; rastering the landing position of the biopolymer jet back-and-forth across the collection solution to make a biopolymer sheet of hydrogel nanofibers having an internal alignment of polymer chains; rolling the biopolymer sheet around a PTFE-coated mandrel in any direction such as perpendicular, parallel, or a mixture thereof forming a wall with a thickness; forming a tubular scaffold comprising a hollow core and one or more sheets comprising hydrogel nanofibers with internal alignment of polymer chains and the tubular scaffold has circumferential, longitudinal, or mixed topography; crosslinking the hollow tubular scaffold by chemical or physical methods; dehydrating the hollow tubular scaffold via lyophilization or graded ethanol treatments; removing the dehydrated hollow tubular scaffold from the mandrel; and forming a dehydrated hollow tubular scaffold. The methods of the present invention may comprise the step of altering the inner diameter of the hollow tubular scaffold by changing the diameter of the mandrel used for wrapping the one or more sheets of hydrogel nanofibers. The methods may also include a step of altering the wall thickness and outer diameter of the hollow tubular scaffold by changing the number of layers of sheets or thickness of the sheet layers comprising hydrogel nanopolymers wrapped around the mandrel. The methods also include a step of crosslinking via either chemical conjugation or physical method to reinforce the scaffold material, which may be followed by a graded dehydration step to generate a dry tubular scaffold for the ease of storage. The dehydrated hollow tubular scaffold may be stored for extended periods at temperature in the range of −80° C. to room temperature, −10° C. to 80° C., 0° C. to 70° C., 10° C. to 60° C., 20° C. to 50° C., or 30° C. to 40° C., for example. A dehydrated tubular scaffold of the present invention may be rehydrated with a reverse graded ethanol treatment and rinse process, for example. A suitable biopolymer jet material used in the present invention may be any polymer used to make a hydrogel nanofiber described above, such as fibrin, for example.

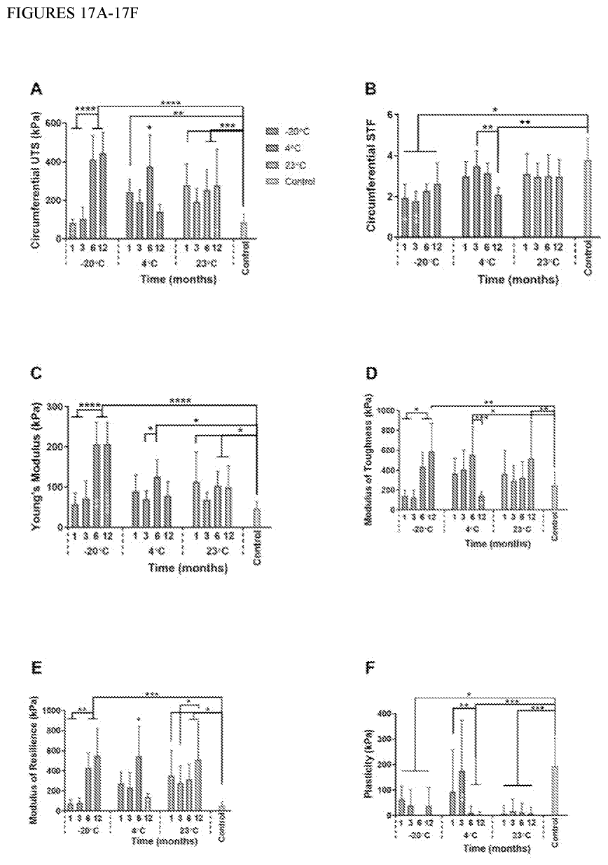

In accordance with a twelfth embodiment, the present invention provides a method of making the tubular scaffold as described above where the crosslinking is accomplished by enzymatic crosslinking with 100 U/ml thrombin with or without Factor XIII in a calcium ion-containing buffer or using other transglutaminase with calcium ions before lyophilization. Alternatively, the tubular protein hydrogel fiber scaffolds can be chemical crosslinked using in 1-ethyl-3-(3-dimethyl aminopropyl) carbodiimide hydrochloride (EDC) with N-hydroxysuccinimide (NHS) dissolved in a buffer to facilitate the crosslinking reaction prior to lyophilization (FIG. 17). Additionally, other typical difunctional or multi-functional chemical crosslinking agents including but not limited to glutaraldehyde, paraformaldehyde, dithiobis(succinimidyl propionate), PEGylated bis(sulfosuccinimidyl) suberate, dimethyl 3,3′-dithiobispropionimidate.

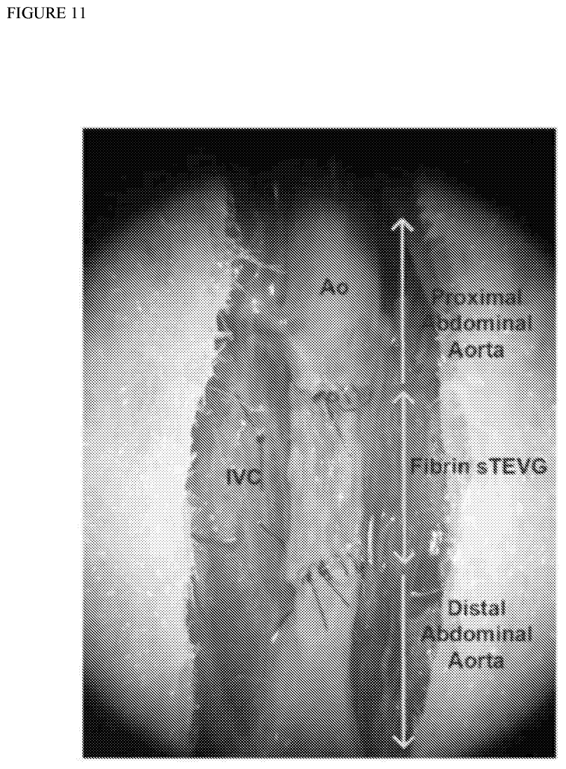

In accordance with a thirteenth embodiment, the present invention provides a method of treating a vascular injury or disease in a subject comprising the steps of: extracting cells from a subject with a vascular injury or disease; providing a perfusion bioreactor of the present invention, first seeding cells of the subject into the perfusion bioreactor on day 0; one or more seedings of the same or different cell types of the subject to allow a confluent cell layer or multiple layers of cells to form on the tubular scaffold; culturing to form a microvascular structure having a hollow core; and implanting the microvascular structure having a hollow core at the site of the vascular injury or disease of the subject (FIG. 11).

Biodegradable Tubular Microfibers

The inventors used biodegradable microfibers (described in Zhang, et al., 2014 and U.S. Pat. No. 10,119,202 B2, both incorporated by reference into this patent application) to create tubular scaffolds and sTEVGs. Briefly, solid fibrin microfibers are fabricated by electrospinning a fibrin biopolymer jet onto a thrombin collection solution. Microfiber bundles of varying diameters are collected and dehydrated to create the solid microfiber with controlled outer diameters. Dehydration can include freezing and lyophilization or graded ethanol treatments. These solid microfibers may have alignment of their surface topography, which can be used to culture cells. Once the fibers have been cellularized with the desired cell populations, the solid microfiber fibrin core can be degraded with plasmin to create a hollow structure with cell walls. The sTEVGS of the present invention have been successfully implanted into mammals and may be used to treat vascular disease such as pediatric CCD, CAD, or PAD.

Microvascular Development Protocol in Three-Chamber Bioreactor

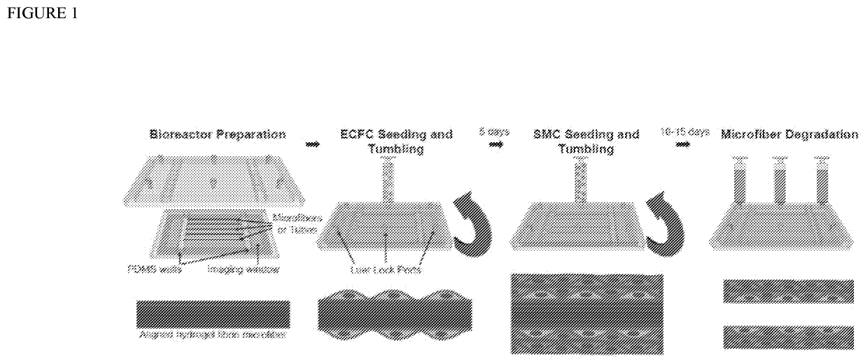

Following the inventor's recent success in creating self-standing luminal multicellular microvascular structures in vitro utilizing fibrin hydrogel microfibers as molds for structure development (Barreto-Ortiz, et al., 2015), the inventors designed a bioreactor device that would allow complete control over culture conditions while having built-in capabilities to perfuse the microvessel. The inventors designed a bioreactor comprised of two polycarbonate plates with a single rectangular chamber inside to achieve this goal (FIG. 1). This space was divided into three separate compartments by two PDMS walls, which also served as anchors, to which a fibrin microfiber was tethered. Each of these three compartments featured two luer lock ports to allow controlled media changes and perfusion. Using this bioreactor and following their recently published protocol (Barreto-Ortiz, et al., 2015) the inventors devised a step-wise process for the generation of an in vitro microvessel model (FIG. 1).

The bioreactor preparation consisted of placing two PDMS walls in the bioreactor compartment and tethering the fibrin hydrogel microfibers in between the two walls before sealing and sterilizing the complete system. ECFCs were then seeded on the hydrogel microfibers by adding a cell suspension in the middle chamber and tumbling overnight to optimize cell attachment. ECFCs were cultured for 5 days to allow a confluent endothelial layer to form, after which vSMCs were seeded on top in a similar manner Culture was continued for 10 to 15 more days to obtain a robust tunica media. After this, the system was treated with a plasmin solution to degrade the fibrin hydrogel microfiber core of developing microvessel grafts.

Three-Chamber Bioreactor Design and Implementation

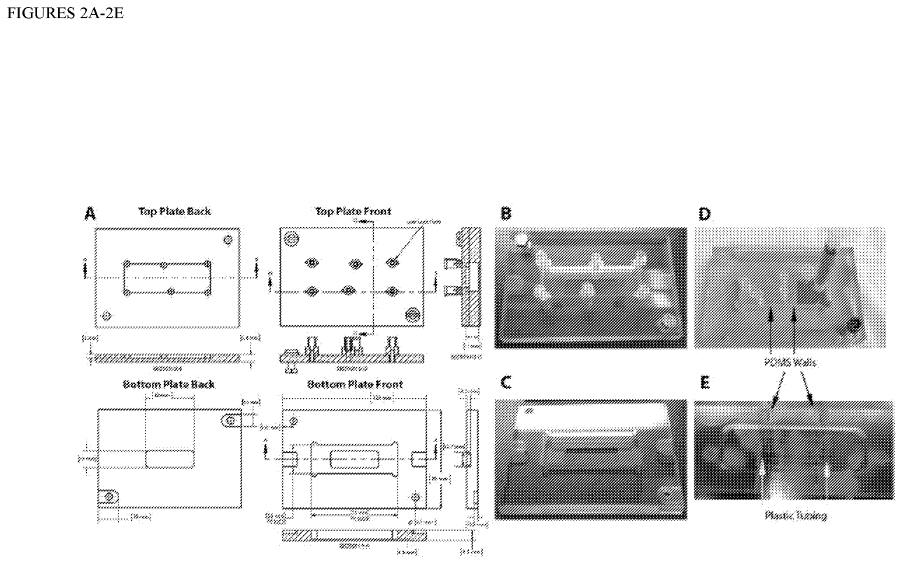

Two different bioreactor prototypes were designed and tested, each composed of two symmetrical plates enclosing three compartments: a middle compartment to hold the microfibers and serve as the seeding and culture chamber and two outer compartments to serve as inlet and outlet media ports for perfusion. The final bioreactor was designed to have the same dimensions as a standard cell culture well plate (125×85 mm) and contain an imaging window in the bottom plate (42×15 mm) within an inner compartment fitting a standard microscopy glass slide (75×25 mm), as shown in FIGS. 2A-C. This enabled live imaging of developing structures within the device, providing the opportunity for detailed monitoring. The bioreactor plates were designed to be held in place by two standard jackscrews and nuts, and vacuum grease was used to seal the microscopy slide to the bottom plate as well as to seal the two plates together. Two indentations were fabricated on each side of the bottom plate to allow leverage between the plates when dismantling the device.

The inner chamber was designed to have a total height of 11 mm, 8 mm within the bottom plate and 3 mm from the top plate (FIG. 2A). A total of 6 Luer lock ports were built in to allow individual media changes within each of the three separate compartments created after the PDMS walls are placed within the chamber (FIGS. 2A-D). As shown in FIG. 2D, this can be done without leaking between the 3 chambers, allowing for detailed control of seeding and culture conditions. Up to five fibrin microfibers were tethered in between the PDMS walls in the middle compartment of the inner chamber by feeding them through plastic tubing traversing each PDMS wall (FIG. 2E). This design would allow the developing cell wall to grow both on the outer surface of the microfiber and, near the PDMS walls, also on the outer surface of the tubing, creating a self-cannulating system for the developing microvasculature (data not shown).

Development of Microvascular Structures with a Tunica Media

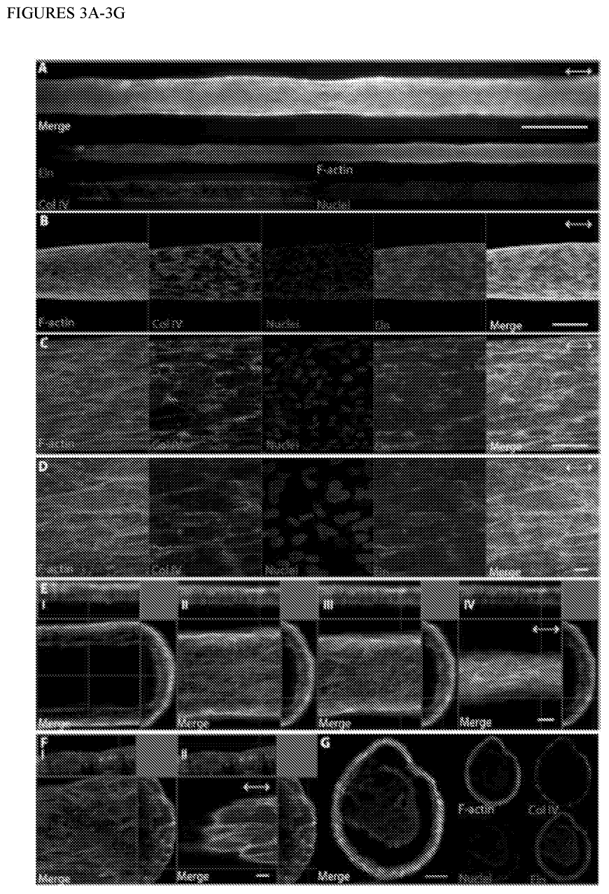

Previously the inventors showed that fibrin microfibers seeded with ECFCs for 5 days and co-cultured for 5 more days with vSMCs created a multicellular structure with a fully invested mural cell layer (Barreto-Ortiz, et al., 2015). However, native microvasculature larger than 250 μm in diameter has a tunica media of at least 3 layers (Mulvany, et al., 1978). In the current invention, the inventors cultured vSMCs on ECFC-seeded microfibers for 10-15 days to achieve a multi-layer tunica media before treating the fibrin microfiber core with plasmin for degradation. As shown in FIG. 3A-D, the resulting structures were uniform throughout their full length and express necessary ECM proteins for vascular stability, including Col IV and elastin.

Orthogonal projections of confocal images showed structures with a circular cross-section, and a vessel wall of 57.8±3.9 μm and inner diameter of 185±8.2 μm (FIG. 3E-F). Furthermore, these projections revealed the presence of multiple cell layers, starting with a confluent aligned cell layer at the inner wall (FIG. 3E II), a layer with cells exhibiting diagonal projections in the middle (FIG. 3E III, FIG. 3F I), and a confluent aligned cell layer in the outer part of the cell wall (FIG. 3 E IV, FIG. 3F II). Cross-sectional cryo-sections of these structures showcased the circular cross-section of the structures, robust cell wall, and marked deposition of ECM proteins (FIG. 3G). However, the development of a multilayered robust tunica media created a new challenge for complete microfiber degradation with some fibrinolysis products remaining inside the structure's lumen even after plasmin treatment, leading to the development of hollow, fibrin microfibers. The development of microvascular structures occurred step-wise and could be precisely controlled at each step.

Fabrication and Manufacturing of Tubular Scaffolds Comprising Hydrogel Nanofibers Having a Hollow Core

In microvasculature, it has been shown that vessels collapse in the absence of flow (Berger and Jou, 2000). Indeed, the inventors observed structures would begin losing patency after 10 or more days following coculture with vSMCs, even without complete fibrin microfiber degradation. Longer culture time points resulted in complete diameter reduction and structure failure, and perfusion through developing structures was obstructed by remaining fibrinolysis products. In order to have better control over perfusion and prevent lumen occlusion, the inventors developed hollow fibrin hydrogel tubes with the same aligned nanotopography and bioactive substrate as their fibrin microfibers to give rise to tubular scaffolds that are hollow and used to create sTEVGs. WO 2013/165975, U.S. Pat. No. 10,119,202 B2, (incorporated by reference) discloses a manufacture of solid hydrogel microfibers. Here the inventors hypothesized that the hollow space of these tubes could be used to apply flow through developing structures at earlier stages of microvascular development, independent of fibrin degradation, thus preventing structure collapse.

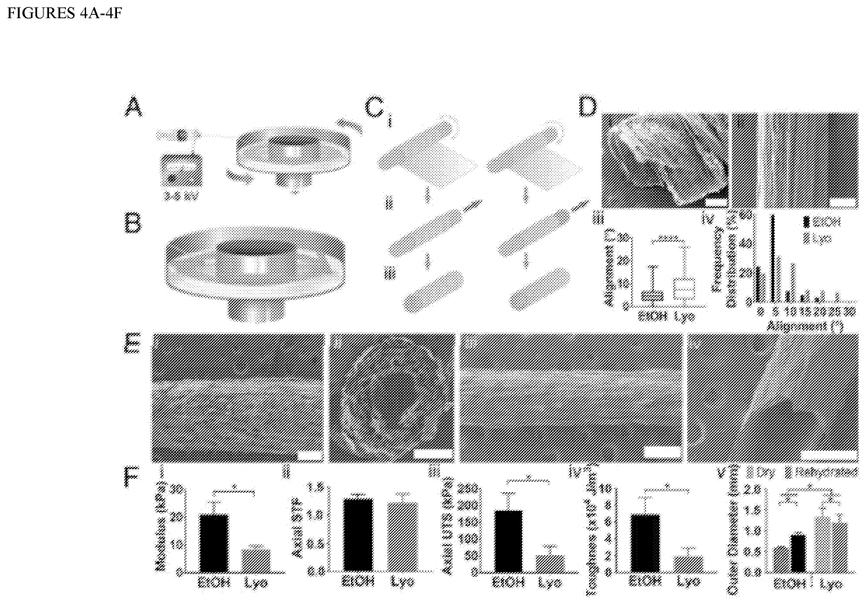

Tubular scaffolds of the present invention including hydrogel nanofibers are made by a different process than hydrogel microfibers described in U.S. Pat. No. 10,119,202 B2. Instead of creating a bundle of hydrogel nanofibers having polymer chains with an internal alignment, the fibrin hydrogel nanofibers are spun into a sheet by rastering the landing position of the fibrin biopolymer jet back-and-forth across a thrombin collection solution. To create hollow microfibers, the inventors fabricated tubular structures by wrapping the aligned fibrin sheets on PTFE coated mandrels (FIGS. 4A-B) (Zhang, et al., 2014). Wrapping the fibrin sheets with their alignment perpendicular to the mandrel's axis resulted in a circumferentially oriented nanotopography on the hollow microfibers, while keeping the fibrin sheet's alignment parallel to the mandrel's axis resulted in longitudinally aligned nanotopography (FIG. 4C). Other angles of fiber orientation may be achieved by altering the angle of the PTFE mandrel relative to the sheet alignment. The resulting alignment was confirmed with SEM for both circumferentially (FIG. 4D I) and longitudinally aligned (FIG. 4D II, EI-IV) microfibers. Detail of cross-sectional morphology are shown in FIG. 4D After wrapping, microfiber sheets were frozen and lyophilized to ease removal of the mandrel from resulting hollow fibrin microfibers. Lyophilized tubular scaffolds had a highly internal porous structure following removal of water but tube walls were very thick (FIG. 4E I-II). Dehydration with ethanol followed by air-drying reduced wall thickness from several hundred μm down to −100 μm (FIGS. 4E III-IV, F IV). The revised dehydration process using ethanol gradient resulted in reduced tube diameters in both the dry and rehydrated state (gradient drying: 592.4±17.1 μm and 898.7±41.4 μm; lyophilization: 1329.2±82.4 μm and 1186.9±120.6 μm, respectively), improved tube elastic moduli and ultimate tensile strength (gradient drying: 20.9±4.5 kPa and 185.0±51.1 kPa; Lyo: 8.3±1.3 kPa and 51.1±27.5 kPa, respectively), and maintained similar longitudinal strain to failure relative to the lyophilized tubes (gradient drying: 128.8±8.5%; Lyo: 122.2±15.5%) (FIG. 4F). Rehydrated EtOH-dried fibers show increased surface texture (G I) and microfiber density (G II III) as a result of tube swelling upon rehydration.

Endothelial colony forming cells (ECFCs) adhere to microfibers used in the present invention (described in Zhang, et al., 2014 and U.S. Pat. No. 10,119,202 B2, hereby incorporated by reference) that have structural characteristics that align the ECFCs in the direction of flow to form a microvascular structure. Other cells such as mural cells may be random, longitudinal, or circumferentially aligned. A biodegradable tubular microfiber used in the present invention has a longitudinally aligned nanotopography comprising biodegradable, electrostretched hydrogel polymer fibers with internal polymer alignment. The term “longitudinally aligned” nanotopography means the nanofibers are aligned longitudinally with each other within a microfiber along the length of the structure. The term “internal alignment” means the polymer chains in a nanofiber are aligned by mechanical and electrical methods. The “longitudinally aligned nanotopography” means the structure of a microfiber resulting from the “longitudinally aligned” nanofibers having polymer chains with “internal alignment”.

Tubular scaffolds of the present invention are made of the hydrogel nanofibers used to form the microfibers discussed U.S. Pat. No. 10,119,202 B2. However, the tubular scaffolds of the present invention have a different structure then the U.S. Pat. No. 10,119,202 B2 microfibers. Specifically, the microfibers described in U.S. Pat. No. 10,119,202 B2 bundle hydrogel nanofibers to form a solid, without a hollow core, microfiber. The tubular scaffolds of the present invention form a sheet of hydrogel nanofiber, in some embodiments the nanofibers are longitudinally aligned and in other embodiments the nanofibers are random, circumferential, or otherwise angled in a controlled manner, to form a sheet. One or more sheets of these nanofibers is then formed into a cylinder shape having a hollow core creating a tubular scaffold of the present invention. Each sheet may have a separate and distinct alignment of nanofibers in any angle. Some sheets may be random and others aligned. Consequently, the tubular scaffolds of the present invention are designed to novel structures with specific axial and radial strength, and circumferential or longitudinal topography, by layering sheets having the same or different angle alignments or no angle alignment. The tubular scaffolds of the present invention are then used in vascular grafts such as sTEVGs having structural characteristics not seen before.

Other characteristics of tubular scaffolds include in some embodiments that they clearly exhibit longitudinally aligned nanotopography resulting from the bundling of aggregated polymeric nanofibers with internal polymer chain alignment. In addition, these electrostretched hydrogel tubular scaffolds used in the present invention are mechanically stronger and easier to handle than typical hydrogels of the same composition and dimensions and the electrostretched hydrogel tubular scaffolds exhibited preferential alignment along the nanofiber axis. When the crosslinking mechanisms are compatible, multi-component hydrogel tubular scaffolds can be produced with similar degree of alignment. In addition, the nanofibers making up the tubular scaffolds have a highly porous and aligned surface texture (polymer chains are internally aligned) that is also very different from recently developed fibrin microthreads, which are dense and smooth on the surface.

The inner diameter of the tubular scaffold with a hollow core can be altered and controlled by changing the diameter of the mandrel used. Multiple layers of sheets can be wrapped around the mandrel to modulate tubular scaffold wall thickness and resulting outer diameter. Additionally, the direction of wrapping can be altered so that the inner layers of the graft have a longitudinal or circumferential alignment, while the outer layers have a circumferential or longitudinal alignment. This can aid in internal and external cellularization of the fibers, enabling control of cellular orientation in both areas. The tubular scaffolds are then dehydrated via lyophilization or graded ethanol treatments and removed from the mandrel yielding hollow fibrin tubular scaffolds with longitudinal, circumferential, or mixed alignment.

The dehydration of these tubular scaffolds with solid and hollow cores allows them to be stored for extended periods at room temperature or in a fridge (4° C.). While dehydrated or after rehydration, these tubular scaffolds can be shipped to laboratories, hospitals, or other facilities for use to create microvascular structures using the cells of a subject who is suffering from vascular injury or cardiovascular disease. These tubular scaffolds can also be shipped for immediate implantation in a subject who has a severe, emergency condition requiring immediate surgical intervention. Transport in a dehydrated state or in a rehydrated state with sterile solution is possible. In either state, the hollow microfibers can be cannulated on a mandrel or needle. The solid and hollow tubular scaffolds can also be shipped independent of cannulation or other supporting structures in a vial, test tube, plate, well, dish, or other closed container. To facilitate culture, tubular scaffolds can also be shipped in a sterile bioreactor, which would enable the shipment of cellularized or acellular vascular grafts for culture or implantation purposes. Before implanting acellular structures or creating cellularized microvascular structures for implantation or study, the tubular scaffolds should be rehydrated in a reverse graded ethanol treatment. The ethanol treatment further sterilizes the fiber and slowly begins the rehydration process. The fiber is slowly moved to PBS solutions with decreasing ethanol concentrations and rinsed several times to ensure removal of all ethanol before use. After rehydration, the tubular scaffolds with solid and hollow cores are ready for use.

Implementation of Hydrogel Microfibers in Single-Chamber Bioreactor

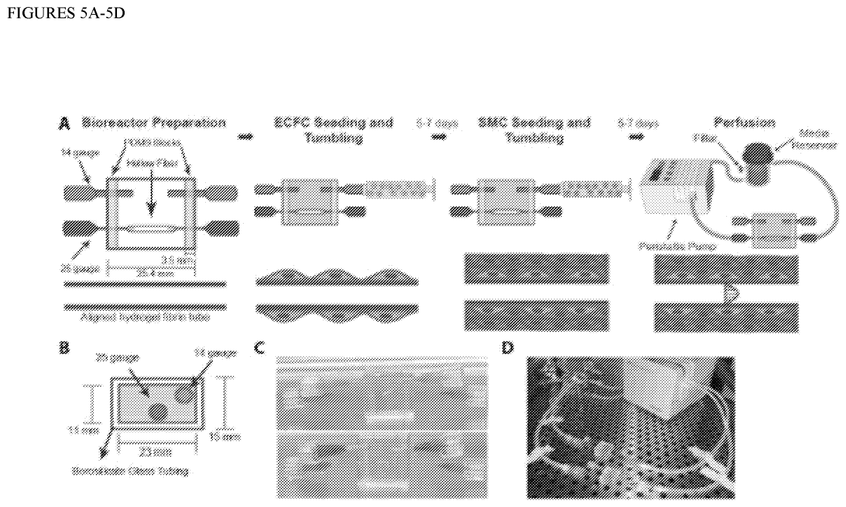

sTEVGs were prepared by growing cells around tubular scaffold of the present invention. This process required a custom bioreactor to encase the developing microvascular structures and support perfusion. For this, the inventors designed a simple yet effective single chamber bioreactor composed of rectangular borosilicate glass tubing capped on both ends with custom fitted PDMS walls. These walls could be traversed with large diameter luer lock needles to create media change ports and with small gauge needles to cannulate and perfuse the tubular scaffolds from day zero, without the need to degrade the fibrin core (FIGS. 5A-B).

To assemble the bioreactor, PDMS walls were first custom cut to fit the 11×23 mm inner rectangular cross-section of the chamber. Then, 14- and 25-gauge needles were punctured though the top and bottom corners of the PDMS, as shown in FIG. 5B. After all needles were in place, one PDMS wall was placed on one end of the glass chamber and the microfiber was cannulated between the two 25-gauge needles and secured with sutures before closing the second PDMS wall. The chamber was then flushed with ethanol and washed with water or PBS before seeding. Final assembled single chamber bioreactors with microfibers cannulated in between two needles are pictured in FIG. 5C. As shown here, the needles were capped with standard luer lock caps until use, efficiently creating a sealed space within the chamber.

This new system enabled the step-wise development of vasculature (FIG. 5A), starting with the seeding of ECFCs on a tubular scaffold of the present invention and tumbling the device overnight to optimize cell attachment. After 5-7 days, vSMCs were seeded on top in the same manner and further cultured before perfusion. With this system, there is no need to apply a plasmin degradation treatment since a solid hydrogel microfiber described in U.S. Pat. No. 10,119,202 B2 is not used, and tubular scaffolds of the present invention can be perfused at any time point by attaching the needles cannulating a tubular scaffold to a closed loop flow system powered by a peristaltic pump (FIG. 5D). Vascular grafts of varying lengths and diameter could be cultured in this bioreactor system by altering the length and gauge of the needle, respectively, used to cannulate the fibrin tubular scaffold.

Fabrication of Perfusable Microvascular Structures or Grafts using Tubular Scaffolds

The feasibility of immediately beginning to perfuse or implant the sTEVG by perfusing the acellular tubular scaffolds with the pulsatile perfusion system at various physiologic shear stresses was also assessed. To definitively reveal any perfusion issues, PBS with blue dye (dye concentration: 6.78 ppm) was flowed through the fiber, while clear PBS (dye concentration: 0.00 ppm) was used to fill the chamber, which would drastically change color from leaks. Perfusion for 24 hours at 7 dyn/cm² or 14 dyn/cm² was successful, with no leaks from the fiber. After 24 hours of high shear stress exposure, the PBS filled chamber was only tinted blue. There was no significant difference in the absolute dye concentration in the chamber between the low and high shear stress conditions until the 24-hour timepoint (0.160±0.020 ppm and 0.200±0.035 ppm, respectively). The uniform distribution of blue tint in the chamber and the small overall increase in absolute dye concentration after 24 hours showed that only diffusion through the fibrin graft wall occurred and flow could be maintained. With this new fibrin hollow tubular scaffold (or tubular scaffold), there was no need to apply a plasmin degradation treatment and structures could be perfused at any time by attaching the needles cannulating the microfibers to a closed loop flow system powered by a peristaltic pump. Thus, immediate, continuous, and controlled perfusion could be applied without damage to the acellular sTEVG. Additionally, there was no significant difference in the absolute dye concentration in the chamber between the acellular and cellularized conditions after 24 hours of perfusion at 7 dyn/cm² (0.160±0.020 ppm and 0.194±0.069 ppm, respectively). Therefore, no loss of graft functionality occurred with adding cells to the natural scaffold of the present invention and lumen patency of the sTEVGs was maintained

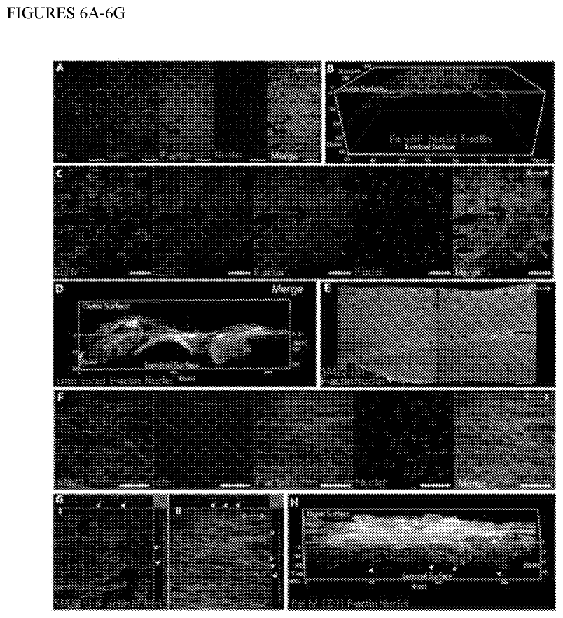

The inventors were uncertain whether solid hydrogel microfibers described in U.S. Pat. No. 10,119,202 B2 would provide similar results to the tubular scaffolds of the present invention, given the previous dependence ECM orientation on curvature and the significant increase in curvature for the tubular scaffolds, so they performed the following test. Since the tubular scaffolds had a larger surface area due to an increased outer diameter, the inventors increased ECFC seeding concentration and culture time to maximize cell coverage. Due to this increased surface area, performing two rounds of cell seeding of ECFCs on day 0 and 4 enhanced the formation of a confluent endothelial layer. Given the increased surface area of the tubular scaffolds made out of the microfibers, altering the length of the microfiber used had marked effects on the attainment of a confluent endothelium (data not shown). After 5-7 days of ECFC culture on tubular scaffolds, the formation of an aligned, confluent endothelium expressing mature endothelial markers vWF and CD31 was observed (FIG. 6A-D). In congruity with their previous results, a diameter of over 450 μm resulted in random deposition of ECM proteins Col IV and Fn by ECFCs (FIG. 6A, C).

vSMCs seeded on top of ECFC-seeded tubular scaffolds and cultured for 5-7 more days resulted in fully invested microvascular structures expressing vSMC marker SM22, EC marker CD31, and evidencing both Col IV and elastin deposition (FIGS. 6E-H). Similarly, to the ECFCs, performing multiple rounds of vSMC seedings every two days resulted in improved multi-layered cellular constructs. At this time point, the structures contained two distinct cell layers, with only the outer cell layer expressing SM22 and elastin (FIG. 6G).

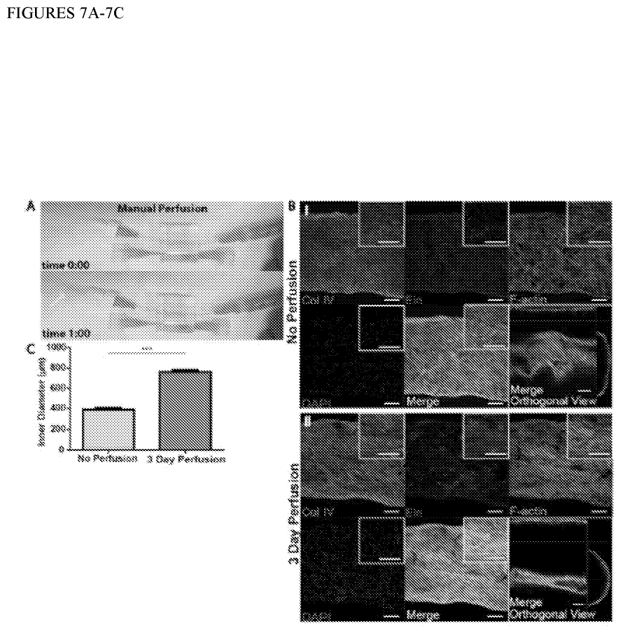

The inventors then verified the perfusion capability of this new system by culturing ECFCs for 5 days followed by co-culture with vSMCs for 5 more days, after which samples were either maintained in static culture conditions for 3 more days or perfused at 5 mL/hour (about 5 dyne/cm²) for 3 days. As observed in FIG. 5D and FIG. 7A the structures could be either attached to a peristaltic pump for continuous long-term perfusion or manually perfused, and perfusion can be visualized following the medium flow from the inlet to the outlet port (FIG. 7A). Interestingly, the inventors observed higher elastin deposition after 3 days of perfusion compared to static control, as well as a more aligned Col IV and F-actin organization (FIG. 7B). More importantly, the lumen of microvascular structures under static conditions started collapsing while perfused developing microvasculature maintained a cylindrical cross-section and a diameter larger than its static counterpart (FIGS. 7B-C). As in the three-chamber bioreactor, the development of cellularized tubular scaffolds occurred stepwise and could be precisely controlled at each step.

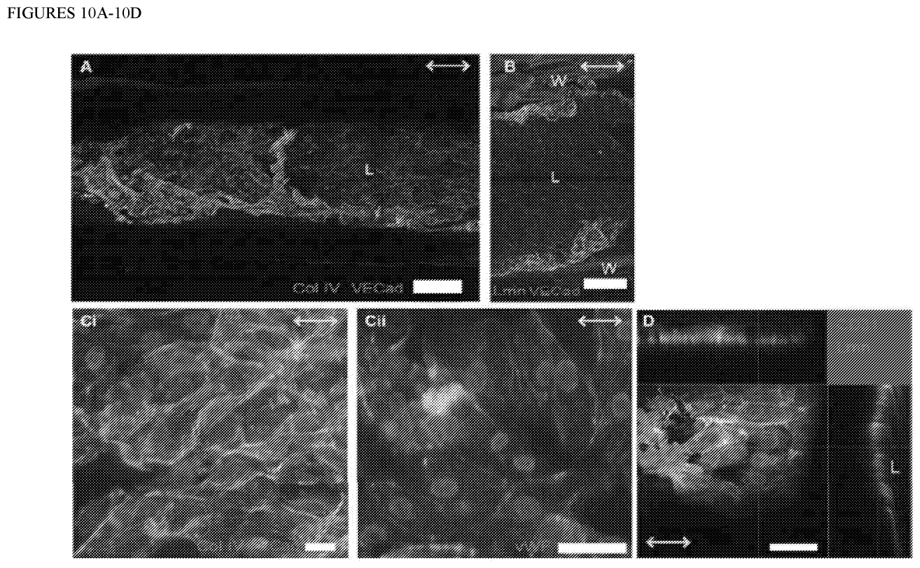

To enhance the hollow tubular scaffolds of the present invention for implantation, we began to internally seed endothelial colony forming cells (ECFCs) onto the grafts via injection into the luminal space. After 3-4 days of static ECFC culture in tubular scaffolds (Internal, Static), the formation of an aligned, confluent endothelium expressing the mature endothelial markers: vWF, VECad, and CD31 was observed (FIG. 10). The fibrin wall of the tubular scaffold was overexposed in FIG. 10B to clearly demonstrate the cellular attachment to the luminal surface. By seeding the cells via injection into the luminal space or by dropwise seeding on the external graft surface, the seeding became more efficient, requiring both fewer culture days to attain a confluent endothelium and fewer cells to be seeded on day 0.

Recently the inventors achieved for the first time the generation of self-standing luminal multicellular microvascular structures or grafts such as sTEVG in vitro. However, the final step in the blood vessel maturation process requires the presence of luminal flow. Most groups that have attempted to create microvascular structures or grafts with perfusion have done this by creating void-space channels within a hydrogel or microfluidic device (Khan, et al., 2011, King, et al., 2001, Zheng, et al., 2012). However, none of these are self-standing or has demonstrated the development of both a tunica intima with a continuous endothelium, a robust tunica media, and their corresponding organized ECM proteins. Studies that have presented perfusion of tissue engineered blood vessels have done so in structures above 3 mm in diameter (Niklason, et al., 2001, Williams and Wick, 2004), with perfusable microvasculature below 3 mm remaining a challenge. Similarly, significant advancement has recently been made in the realm of acellular vascular grafts, but most of these vascular grafts have been larger than 4 mm in diameter (Fukunishi, et al., 2016, Syedain, et al., 2016).

Previous work by Neumann et al. described the use of a perfusion chamber wherein nylon strands were enclosed and used to support the growth of vSMC sleeves in this size range for 21-28 days, after which the nylon strands were mechanically removed, creating perfusable vSMC structures (Neumann, et al., 2003). While this approach allowed for the creation and perfusion of vSMC sleeves in vitro, it did not permit the formation of an aligned endothelium, possibly due to inner cell layer damage upon nylon strand removal (Neumann, et al., 2003). The formation of a confluent endothelium is critical for the reduction of thrombosis risk in vivo and has been a significant focus for small diameter vascular graft development, especially as decreasing diameter is correlated with increased thrombus formation (Brisbois, et al., 2015, Fleser, et al., 2004, Pashneh-Tala, et al., 2015, van Hinsbergh, 2012). For this reason, our bioreactor designs focused on the formation and maintenance of a robust cell wall with a confluent endothelium.

The inventors' established hydrogel fibrin microfiber system allowed the inventors to study growing microvasculature, which was key in understanding the biochemical and biomechanical cues that guide endothelial cell alignment, vascular smooth muscle cell investment, and organized ECM deposition by each cell type. However, longer culture time points were required to develop a multilayered tunica media; and constructing a perfusion system to support nascent vasculature was critical. To be able to extend the culture time of developing structures, the inventors engineered a custom three-chamber bioreactor with specific design features enabling the controlled, progressive fabrication of microvasculature starting with the inventors' established fibrin microfibers.

The newly developed bioreactor allowed up to five structures to be grown at the same time, and in situ monitoring through its imaging window permitted the inventors to observe the formation of the microvasculature and optimize culture conditions and time points in order to achieve a robust microvessel wall. Developing structures could also be fixed, stained, and imaged either inside or outside of the device, allowing the analysis of both cell and ECM markers in the developing structures. As such, structures cultured with ECFCs for 5 days followed by vSMC seeding and culture for 10 to 15 more days evidenced a multilayer, multicellular microvascular structure with a robust expression of ECM proteins Col IV and Eln.

Following the inventors previous microvessel model protocol, the inventors used plasmin to degrade the fibrin core in order to obtain a luminal vessel. Previous results demonstrated this degradation was feasible in structures developed by culturing ECFCs for 5 days followed by 5 days more of vSMC co-culture, a time point where the structures had one layer of each cell type. In the new bioreactor of the present invention, cryo-sections performed on structures cultured with vSMCs for 10-15 days and treated with plasmin for 24 hours revealed a circular lumen and thick vessel wall, but also evidenced remaining fibrinolysis products within the lumen that were not able to fully degrade. As such, longer culture time points after vSMC seeding resulted in more robust microvascular structures, but presented an issue for full fibrin microfiber degradation as the plasmin had to diffuse through a thicker vessel wall to have an effect on the fibrin core. Similarly, fibrinolysis products now had a barrier to diffuse through, and accumulation inside of the vessel wall would prevent flow. Further attempts to strengthen the degradation protocol, including performing several rounds of degradation treatments, were ultimately unsuccessful in completely dissolving the fibrin core (data not shown).

In developing microvasculature, vessels will collapse in the absence of flow. In the inventors' system, the inventors observed partial lumen occlusion, likely caused by increased cellular weight and vSMC contractility, in structures that had been in culture with vSMCs for more than 15 days and that had partial microfiber degradation. Without full degradation, flow was unachievable, and these structures collapsed. In contrast, degrading the microfiber core at earlier time-points resulted in structures that had a distinct lumen but were not robust enough to withstand flow. Therefore, controlling the time-point for perfusion became of critical importance to maintaining lumen stability.

The hollow tubular scaffolds of the present invention are made of nanofibers described in U.S. Pat. No. 10,119,202 B2 and have been demonstrated to surprisingly maintain their longitudinal nanotopography even after being wrapped around a mandrel. However, the tubular scaffolds of the present invention are structurally different from the microfibers described in U.S. Pat. No. 10,119,202 B2. The tubular scaffolds of the present invention are made of sheets of hydrogel nanofibers having internal polymer alignment. The nanofibers of a sheet may be circumferentially aligned or aligned in other angles and one or more sheets may be used to make tubular scaffolds having specific suturability and/or strength depending upon the alignment of nanofibers in a sheet, Furthermore, the wall thickness of a tubular scaffold can be easily controlled by varying the number of sheets made of hydrogel nanofibers, including fibrin, that is wrapped around the mandrel and lumen size can be controlled by changing the mandrel diameter.

In order to create vascular constructs using the tubular scaffolds made of hydrogel nanofibers, the inventors designed a new single chamber bioreactor composed of a glass rectangular chamber sealed on each side by PDMS blocks. The PDMS blocks were punctured with two 14-gauge needles to act as media changing ports and two small diameter needles to cannulate the developing microvascular structures. This allows different size vessels to be generated by simply changing the needle gauge, which also act as the connecting ports for either manual perfusion with a syringe or constant perfusion with a peristaltic pump. This simple design allowed the fabrication of multicellular microvascular constructs with a preformed lumen that when perfused for three days evidenced distinct circular lumen stability and patency, compared to static controls that experienced significant lumen occlusion. This new system is easy to set-up and allows for in situ monitoring of the developing structure. The current prototype allows for the development of one structure per bioreactor at a time. However, several devices can be run concurrently.

Using this new setup, the inventors achieved for the first time the generation of perfusable, self-standing multicellular vascular structures, or grafts such as sTEVGs, in vitro with an aligned endothelium and full vSMC investment. Perfusion can either be done manually for short-term or with a peristaltic pump for prolonged experiments. Furthermore, perfusion can be conducted at any time during the blood vessel development timeline, opening the door for a wide array of 3D flow experiments in a setting recapitulating the cellular and extracellular organization of native vasculature for the investigation of arteriogenesis. As a proof of concept, the inventors demonstrated that perfusion for three days supported lumen stability in developing structures, compared to static culture conditions that resulted in partial lumen occlusion in a fully cellularized construct having ECFCs and vSMCs.

The inventors established a three-compartment bioreactor system and culture protocol, which can be used to generate multicellular microvessels with a robust endothelial vessel layer supported by a fibrin hydrogel microfiber scaffold. The developed construct showed enhanced deposition of ECM proteins Col IV and Eln, as well as a vessel wall composed of three different cell layers. This system allowed structures to be developed in a controlled environment while enabling in situ monitoring, revealing real-time information about microvessel development, including lumen occlusion caused by increased cellular weight and vSMC contractility in the absence of flow. The inventors also developed a novel hollow fibrin microfiber tubular scaffold platform to make vascular grafts including sTEVGs that encompasses the strengths of hydrogel microfibers, while also allowing early stage perfusion through a developing microvessel in order to prevent lumen occlusion. Using this tubular scaffold platform, the inventors successfully generated perfusable multicellular sTEVGs in vitro recapitulating the cell and ECM organization of native vasculature for the first time.

Implantation of Tubular Scaffold as Vascular Graft Including sTEVGs

Small-diameter tissue engineered vascular grafts (sTEVGs) were made from the tubular scaffolds of the present invention having structural characteristics that mimic topographical and biomechanical features of the ECM. Their tubular scaffolds were prepared from hydrogel nanofibers, such as fibrin and exhibits a microscale, longitudinally and/or circumferentially aligned, surface microtopography and tunable stiffness. These longitudinally aligned fibrin nanofibers were prepared to form a hollow, tubular scaffold, serving as a vascular graft such as a sTEVG matrix template, as discussed previously. Fibrin was chosen to develop sTEVGs as it has been shown to improve elastin deposition, a critical ECM component for sTEVGs. The unique surface topography induces endothelial alignment with increased ECM deposition, as discussed above.

For cellularized sTEVGs, human endothelial colony forming cells (ECFCs) were internally seeded on the sTEVGs and cultured in a bioreactor for 3-4 days before implantation in a SCID mouse as an abdominal aorta interposition graft (FIG. 11). We focused on ˜1-mm diameter microvascular grafts for testing in an infrarenal abdominal aorta mouse model for up to 24 weeks (n=4 for each timepoint). The approximately 1-mm diameter microvascular grafts were used as a testing case to allow efficacy studies in an infrarenal abdominal aorta mouse model, which faithfully recapitulates the process of neovessel integration that occurs in large animals and humans, but over a shorter time course. By starting with the smallest, most challenging size, future scale-up to 1-6 mm diameter vascular grafts will be minimally challenging as increased diameter is correlated with decreased thrombus formation and increased patency.

Importantly, the inventive sTEVG design affords the flexibility to create both cellularized and acellular vascular grafts, depending on the application. Acellular tubular scaffolds can be used as an off-the-shelf product for emergency vascular operations; while cellularized sTEVGs can be manufactured for CCD populations that do not require emergency procedures. In order to improve patency and reduce thrombus formation, endothelial cells will be used as a bioactive component to encourage remodeling of the graft by host cell infiltration. By using acellular and cellularized, bioactive sTEVGs using a natural, fibrin scaffold the negative outcomes associated with conventional synthetic sTEVGs is minimized.

Dehydrated, acellular sTEVGs were stored for several months before use. Grafts were implanted and ruptured on post-operative day (POD) 2±1 due to a tear in the fibrin wall along the longitudinal topography emanating from the sutures (n=5). An improved graft composed of longitudinally and circumferentially aligned microfibers (Multidirectional) resulted in a significantly increased mouse lifespan to POD 9±2 (n=6), but rupture with the same tear propagation pattern from the suture points still occurred. The suture retention strength (SRS) for the longitudinal and multidirectional grafts was 43±6 mN and 49±114 mN, respectively, far below the native mouse aorta SRS of 128±33 mN (n=2). In all cases, the grafts were able to withstand aortic flow for several days; yet, over time, these natural grafts began to fail due to mechanical inadequacies, as the field would suggest. Using a surgical sleeve as an intervention to prevent tearing at the suture sites resulted in a significantly larger SRS of 207±87 mN and survival for up to 24 weeks (n=4).

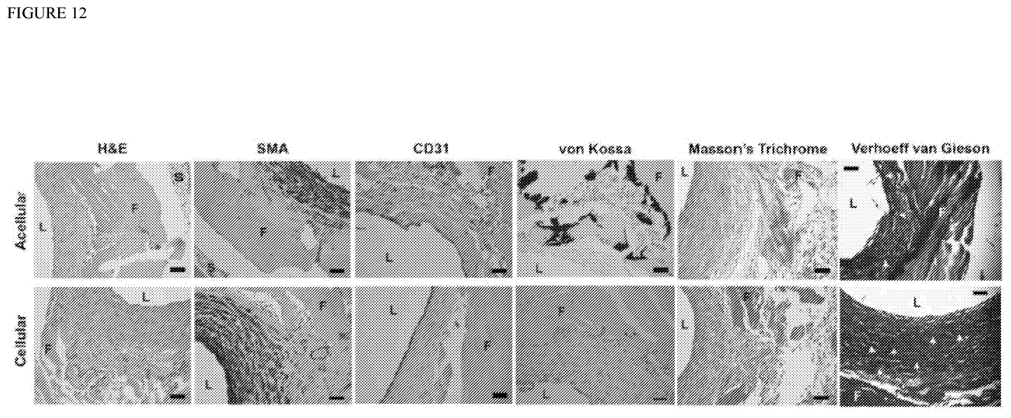

Acellular grafts occasionally developed clots on the luminal walls by week 1, which did not appear in the cellularized grafts. At later time points, no evidence of these clots was visible in either acellular or cellularized sTEVGs, suggesting an antithrombotic benefit of ECFCs was acute. By week 8, significant host cell infiltration could be seen throughout the fibrin sTEVG with delamination and fragmentation of the fibrin (FIG. 12 H&E). The regenerating tissue was densely populated with circumferentially oriented SMA-positive smooth muscle cells (SMCs), which had a confluent, luminal lining of CD31-positive endothelial cells (FIG. 12 SMA, CD31). Von Kossa staining indicated that the acellular graft had increased calcification relative to the cellular sTEVG (FIG. 12). Collagen and elastin staining indicated significant, organized ECM deposition similar to, but not as defined as, the mature native aorta (FIG. 12 MT and VVG). To longitudinally assess graft function, color doppler echocardiography was used to visualize blood flow through the graft and measure blood velocity in the distal aorta prior to the iliac bifurcation. Over 20 weeks, both the acellular and cellularized graft groups had no significant difference in peak systolic velocity, end diastolic velocity, or pulsatility and resistivity indices relative to the baseline native aorta, indicating no change in vascular function due to sTEVG implantation. The inventors fabricated a man-made, hollow fibrin tubular scaffold with controlled surface topography, enabling controlled endothelialization or immediate implantation as a potential candidate for arterial bypass surgery.

Long Term Storage

Off-the-shelf availability of sdVGs is critical to patients needing emergency arterial bypass. Important factors for hospitals focused on value-based purchasing are the storage conditions and product expiration date.⁷ Medical device choices, including items for cardiovascular surgery, highly affect hospitals' supply-chain efficiency and revenue.⁷ To best serve the patient, surgeon, and hospital, it is important to understand the effects of long-term storage on natural polymer-based scaffolds

We studied the effects of long-term storage on the mechanical properties of the sdVGs by using accelerated aging at elevated temperatures in humidified incubators to simulate the storage of sdVGs for 1, 3, 6, and 12 months at ambient storage temperatures of −20° C., 4° C., and 23° C.^{8,9} In terms of circumferential ultimate tensile stress (UTS), the grafts aged in −20° C. became significantly stronger after 6 months of storage relative to earlier time points and controls, which were tested within 5 days of storage (FIG. 17A). Most grafts were significantly stronger than the control grafts after long-term storage. The grafts aged in 23° C. exhibited the most stable UTS and circumferential strain to failure (STF) across all aging times (FIGS. 17A-B). The STF indicates that grafts stored at −20° C. were significantly less deformable than the controls. The grafts at 4° C. became steadily less deformable over time. The Young's modulus indicates grafts at −20° C. became significantly stiffer after 6-12 months of storage (FIG. 17C). At 4° C. and 23° C., the Young's modulus of the grafts was relatively consistent, but the grafts at 23° C. were significantly stiffer than the control grafts. The modulus of toughness, or the energy absorbed by the material until failure, of the −20° C. group had a significant spike from 3 months to 6 months (FIG. 17D). The toughness of the grafts in 4° C. dropped dramatically from 6 months to 12 months. The grafts stored in 23° C. showed no significant changes in toughness over time. The modulus of resilience, or the energy absorbed by the material during elastic deformation, of the grafts displayed very similar trends as toughness for all temperatures (FIG. 17E). However, when resilience was subtracted from toughness to assess plasticity, all stored grafts had decreased plasticity relative to controls (FIG. 17F). The 4° C. grafts aged up to 3 months were more plastic than the grafts aged for 6 months or more. Overall, from this preliminary data, 23° C. storage provided the most stable mechanical properties. While the 23° C. grafts were as tough as the control, this was almost entirely due to elasticity rather than plasticity. Furthermore, the mechanism by which the strength, stiffness, toughness, and resilience increase at −20° C. is unknown but this process can be utilized to fabricate significantly stronger natural polymer grafts and alter sdVG mechanical properties in a controlled manner in a short period of time.

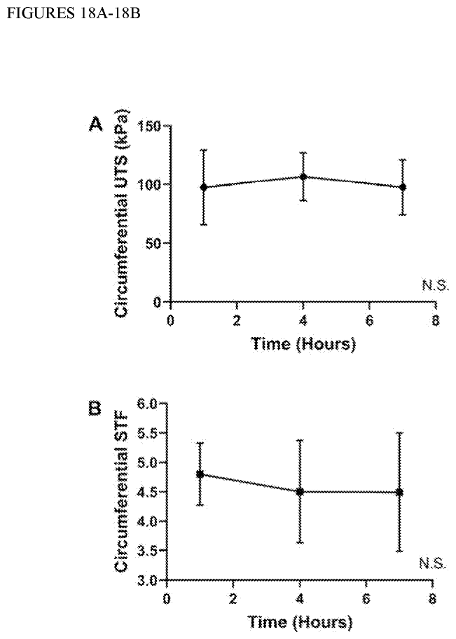

In further studies, it was found that the time of testing after rehydration has no significant effect on circumferential UTS or STF for up to 7 hours (FIG. 18). Therefore, rehydration time prior to use is not critical.

Small diameter vascular grafts (sdVGs) were stored for real time at 1 and 3 months to compare to the accelerated aging study. The storage conditions analyzed were room temperature, a fridge/cold room, and a freezer (Table 1).

TABLE 1
Temperature and humidity conditions for real time storage of sdVGs.
The sdVGs were stored for 1 and 3 months at conditions similar
to those simulated with the accelerated aging study. Temperature
and humidity were recorded at least weekly for each condition.

| | | Real Time | |
|---|---|---|---|
| | Accelerated Aging | Aging | Real Time |
| | Temperature | Temperature | Aging |
| Condition | Estimate (° C.) | (° C.) | Humidity (%) |
| Room | 23 | 21.9 ± 0.8 | 54.3 ± 5.6 |
| Temperature | | | |
| (RT) | | | |
| Cold Room | 4 | 2.6 ± 0.1 | 90.3 ± 0.6 |
| Freezer | −20 | −16.7 ± 2.1 | 85.4 ± 4.6 |

Real Time Storage Data Results

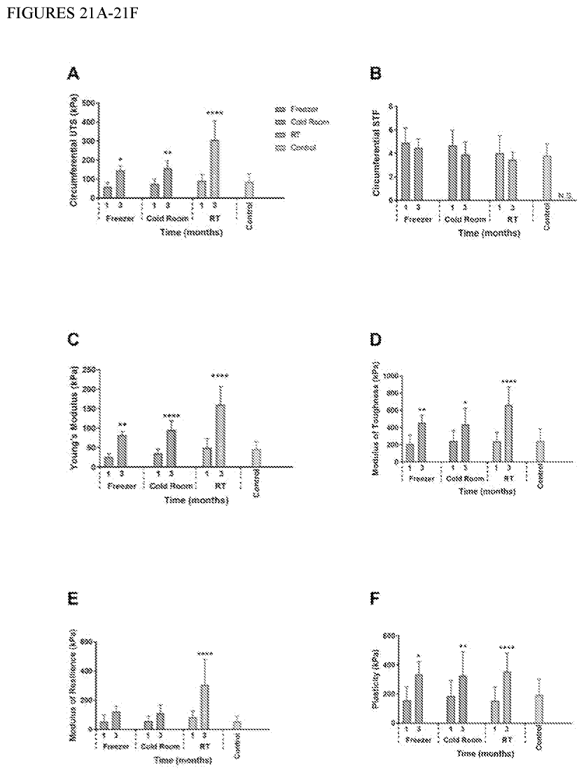

The sdVGs were found to be very consistent after 1 month of storage with the control grafts, which were tested within 5 days of fabrication, for all assessed mechanical properties (FIG. 21). In terms of circumferential UTS, the sdVGs stored for 3 months were able to withstand more stress before failure than the control or 1-month groups (FIG. 21A). The grafts stored for 3 months at room temperature were also able to withstand more stress than those stored in a cold room or freezer. The sdVGs had no significant difference in deformability over time or between storage conditions (FIG. 21B). The stiffness and toughness of the sdVGs had the same trends as the UTS (FIGS. 21C-D). The modulus of resilience only significantly increased for sdVGs stored at room temperature for 3 months (FIG. 21E). The sdVGs stored for 3 months were also more plastic than the control or 1-month groups (FIG. 21F). Ultimately, this study confirms that sdVGs can be stored in a freezer, fridge, or at room temperature for extended periods of time. Further, we have confirmed that the mechanical properties of the hydrogel structure are improving with storage time by becoming stronger and stiffer, towards the properties of native vessels. This shows we can use storage at various temperatures to alter mechanical properties in a controlled manner to more closely mimic the mechanical properties of the native arteries and veins.

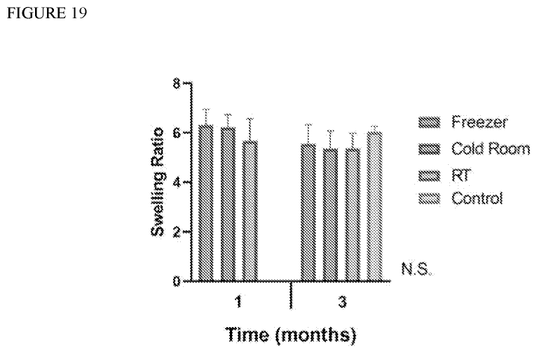

An important assessment for hydrogel structures is the swelling ratio, or ability to absorb water, after storage. For structures that we stored in a freezer, fridge, and at room temperature, there was no significant difference in swelling ratio between the different ambient storage groups (FIG. 19). The swelling ratio also did not significantly change between groups tested immediately after dehydration, after 1 month, or after 3 months of real time storage. Ultimately, storage of the grafts does not affect the swelling ratio of our electrospun hydrogel structure, which is critical for tissue regeneration.

Comparison of Accelerated Aging and Real Time Storage Results:

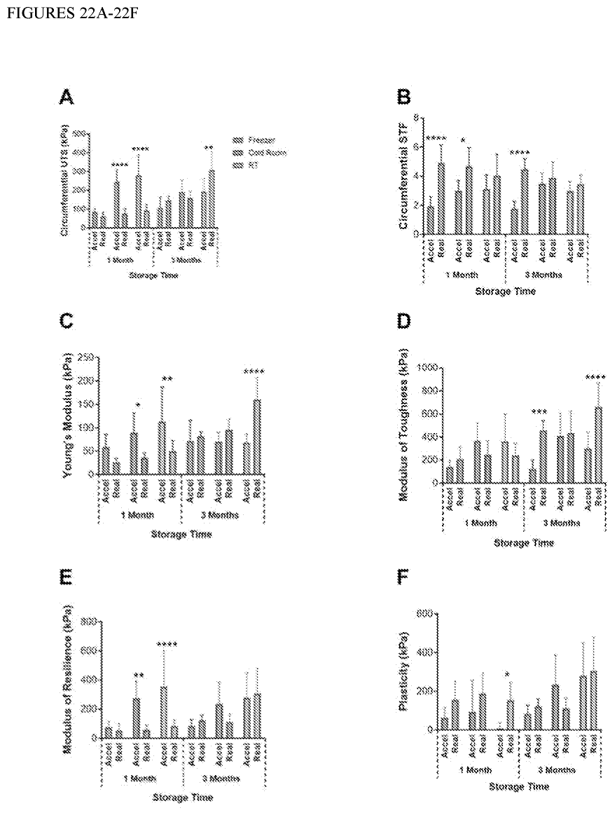

Relative to the control sdVGs, the accelerated aging process predicts similar results to the real time storage in terms of circumferential UTS, elasticity, toughness, and resilience (FIG. 21). Accelerated aging can be used to achieve similar properties or further increase the strength and stiffness of the sdVGs relative to real time storage in less than 1 week (FIG. 22).

Kits of the Disclosure

Any of the compositions described herein may be comprised in a kit. In a non-limiting example, a tubular scaffold of the present invention, that is dehydrated or rehydrated, may be comprised in a kit. The kits may comprise a tubular scaffold of the present invention, preferably a dehydrated tubular scaffold or tube, and a suitable aliquot of one or more reagents to rehydrate the tubular scaffold. Such reagents would include those used in a reverse graded ethanol treatment, for example. The component(s) of the kits may be packaged either in aqueous media or in lyophilized form. The container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the tubular scaffolds of the present invention and any other reagent containers in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly preferred. However, the components of the kit may be provided as dried powder(s). When reagents and/or components are provided as a dry powder, the powder can be reconstituted by the addition of a suitable solvent. It is envisioned that the solvent may also be provided in another container means.

### EXAMPLES

Cell Culture

Cells were cultured in a humidified incubator at 37° C. and 5% CO₂ as previously described (Barreto-Ortiz, et al., 2015). Human ECFCs (Lonza, Walkersville, Md.) were expanded on collagen I (BD Biosciences, Franklin Lakes, N.J.) coated flasks in Endothelial Growth Medium-2 Bulletkit (Lonza) supplemented with 10% fetal bovine serum (FBS; Hyclone, Logan, Utah) and used for experiments between passages 7 and 10. Medium was changed every other day and cells were passaged every 5 to 7 days with 0.05% trypsin (Invitrogen, Carlsbad, Calif.).

Human vSMCs (ATCC, Manassas, Va.) were used between passages 7 and 10 and cultured in F-12K medium (ATCC) supplemented with 0.01 mg/ml insulin (Akron Biotech, Boca Raton, Fla.), 10% FBS (Hyclone), 0.05 mg/ml ascorbic acid, 0.01 mg/ml transferrin, 10 ng/ml sodium selenite, 0.03 mg/ml endothelial cell growth supplement, 10 mM HEPES, and 10 mM TES (all from Sigma-Aldrich, St. Louis, Mo.). Medium was changed every third day and cells were passaged every 5 to 7 days with 0.25% trypsin (Invitrogen).

Preparation of Fibrin Hydrogel Microfibers

Fibrin hydrogel microfibers were fabricated as previously described (Barreto-Ortiz, et al., 2015, Barreto-Ortiz, et al., 2013, Zhang, et al., 2013). Briefly, 1.5 wt % alginate (Sigma-Aldrich) was mixed in-line with 2.0 wt % fibrinogen (Sigma-Aldrich) at flow rates of 2 ml/h and 1 ml/h, respectively. Both solutions were dissolved in 0.2 wt % PEO (Sigma-Aldrich). A 4 kV electric potential was applied to a 25-gauge needle through which the solution was extruded. The resulting jet was collected in a grounded, rotating bath containing a crosslinking solution of 50 mM CaCl₂) with 10 units/ml thrombin (Sigma-Aldrich) for 35 min. Fibers were left in the crosslinking solution for an additional 10 min and then soaked overnight in 0.25 M sodium citrate to remove alginate from the fibrin fibers. Fibers were then washed in DI water for 60 min, bundled and stretched to 150% of their initial length, and air-dried for 60 min.

Preparation of Longitudinally Aligned Fibrin Hydrogel Tubes

Fibrin hydrogel microfiber sheets were prepared similarly to the hydrogel microfibers by electrospinning 2.0 wt % fibrinogen solution co-dissolved in 0.2 wt % polyethylene oxide (PEO) in water under the effects of an applied electric field (4.5 kV) to propel the resultant fiber jet across an air gap of 2 cm and onto a rotating collection bath (45 rpm) containing 50 mM calcium chloride and 20 U/mL thrombin. The landing position of the spinning jet was rastered back and forth via use of a linear stage during the spinning step to yield a uniform aligned fibrin sheet. Hollow fibrin tubes with longitudinal alignment were formed by rolling sheets arranged parallel to the fiber orientation onto polytetrafluoroethylene (PTFE)-coated stainless-steel mandrels to generate tubes. Tube wall thickness was controlled by altering the number of wraps around the mandrel. Following wrapping, fibrin tubes were further crosslinked in 100 U/ml thrombin for 2 h before lyophilization. Alternatively, fibrin tubes were crosslinked for 15 hours in 40 mM EDC/100 mM NHS dissolved in PBS and dehydrated in a series of 25, 50, 60, 70, 80, 90, 95, 100, 100, and 100% EtOH solutions for a minimum of 15 minutes per step and then allowed to air dry. Dried fibrin tubes were removed from the PTFE mandrels following either drying method.

SEM

Dried fibrin tubes (lyophilized or EtOH treated) were attached to conductive carbon tape on metal stubs and then sputter coated with a 15-nm layer of Au/Pd (Hummer 6.2 Sputter System, Anatech UDA, Hayward, Calif.). Samples were imaged using a JEOL 6700F field emission electron microscope at an accelerating voltage of 5 kV.

Mechanical Testing

Fibrin tube mechanical testing for elastic modulus, strain to failure, and axial ultimate tensile stress was done using a Q800 DMA (TA Instruments, New Castle, Del.) under tensile loading conditions in controlled ramp force mode. Hydrated samples were quickly removed from solution and loaded onto the instrument clamps with a preload force of 0.001 N. Tubes were then subjected to increasing force load (ramp rate of 0.05 N/min) until tube failure.

To determine fibrin tube ultimate suture retention strength (SRS), the sample was mounted onto an electromechanical puller (DMT560; Danish Myo Technology A/S, Aarhus, Denmark). After calibration and alignment, the pins were slowly moved apart using an electromotor at a rate of 100 μm/sec to axially pull 10-0 nylon sutures placed through one wall at a distance of 2 mm from the graft end. One suture loop was placed through each end of the graft and then looped over a puller pin.

Three-Chamber Bioreactor Design

Initial 3D designs were created using SketchUp (Trimble Navigation, Sunnyvale, Calif.), while final computer-aided designs were made using SolidWorks (Concord, Mass.). Johns Hopkins Whiting School of Engineering Manufacturing assisted with the final design and manufacturing of the devices.

Three Chamber Bioreactor Preparation and Cell Seeding

Prior to each experiment, the bioreactor components were autoclaved including top and bottom plates, 6 Luer lock caps (Qosina, Edgewood, N.Y., USA), two jackscrews and nuts, and a 75 mm×25 mm glass slide (Fisher Scientific). Two PDMS blocks (1:7 ratio, Sylgard 184, Dow Corning, Midland, Mich.) were cut and fitted to the bioreactor's inner chamber. Vacuum grease (Dow Corning, Midland, Mich., USA) was then applied generously to the bottom of the glass slide and the interface of the two plates, leaving a 1.5 cm barrier around the inner chamber of the device on both plates. All surfaces coated with vacuum grease were left under UV light for 15 minutes. The glass slide was then placed in the inner compartment of the bottom plate and sealed by pressing down firmly across the surface. The PDMS blocks were punctured with a 25-gauge needle and hydrogel microfibers were tethered in between the blocks using polyester shrink tubing (Advanced Polymers, Salem, N.H., USA) through these puncture openings. Fibers were secured in place using 6-0 sutures (Henry Schein, Melville, N.Y.) before sealing the bioreactor. The entire system was sterilized with 75% ethanol and rinsed with sterile water 3 times. After the sterilization process, the exterior compartments were filled with PBS or media and the inner chamber with a cell suspension containing 2×10⁶ ECFCs or vSMCs in ECFC media supplemented with 1% penicillin/streptomycin (Life Technologies). Bioreactors were tumbled for 24 hours to optimize cell seeding and medium was changed every other day thereafter.

Plasmin Treatment

Fibrin hydrogel microfibers with cells were treated with 9 μg/mL plasmin from human plasma (Athens Research & Technology, Athens, Ga., USA) and 2 u/mL alginate lyase (Sigma-Aldrich) in Dulbecco's Modified Eagle Medium (DMEM; Life Technologies) for the time periods specified.

Live/Dead Assay

Samples were incubated with 2 μM calcein AM and 4 μM ethidium homodimer (Invitrogen) in PBS for 30 min at 37° C. and 5% CO₂. Samples were imaged immediately after.

Single Chamber Bioreactor Preparation

Borosilicate tubing (Friedrich and Dimmock Glass, Millvile, N.J.) with dimensions 13 mm×26 mm cut in 38 mm long pieces were cleaned and autoclaved to ensure sterility. Two PDMS blocks were custom cut to each end of the bioreactor. A one inch 25-gauge blunt tip luer lock needle was then punctured 3 mm from the bottom of both PDMS blocks. Similarly, a size 14-gauge blunt tip luer lock needle was punctured through both blocks at the top left corner of each block and capped with luer locks for media changes. The conduit fibers were then cannulated and sutured between the two 25-gauge needles. The bioreactor was sealed before sterilizing with 75% ethanol and washing three times with sterile distilled water or PBS. The needles cannulating the microfiber are capped until perfusion.

ECFCs were seeded on day 0 and 4 in the bioreactor at 5×10⁶ cells in ECFC media supplemented with 1% penicillin/streptomycin (Life Technologies) and 50 ng/mL VEGF (Pierce, Rockford, Ill., USA). Bioreactors were tumbled for 24 hours to optimize cell seeding and medium was changed every other day. vSMCs were seeded on top of the ECFCS 5 to 10 days after ECFC seeding at 1-3×10⁶ cells using a single seeding or repeated seedings every 2 days in ECFC media supplemented with 1% penicillin/streptomycin. Structures were cultured for 5-13 more days before perfusion.

Perfusion

The 25-gauge needles cannulating the microfibers were connected to either a luer lock syringe for manual perfusion or to silicone tubing for perfusion with a peristaltic pump. A media reservoir was used with an air filter to allow gas exchange, and the whole set-up was placed in an incubator. Flow rate was set to 5 mL/hour (equivalent to ˜5 dyne/cm²) or above. Spectrophotometry was used to determine the absolute dye concentration in the chamber after perfusion of fluid with a blue dye (792.8 g/mol) was flowed through the tubular scaffold.

In Vivo Implantation

To internally seed ECFCs on a vascular graft comprising a tubular scaffold for implantation, a cell suspension of 1.4×10³ cells/μL was injected through the bioreactor cannulation needles and into the vascular graft comprising a tubular scaffold comprising fibrin. Subsequently, bioreactors were tumbled for 24 hours to optimize cell seeding. All cellularized grafts were cultured for 3-4 days.

Vascular grafts were implanted as abdominal aorta interposition grafts, as previously. Briefly, anesthetized SCID Beige mice (n=54) (Charles River, Frederick, Md.) was placed in the supine position and opened with an abdominal midline incision. A portion of the infrarenal abdominal aorta was exposed, cross-clamped, and excised. A 3-5 mm length of fibrin nanofiber scaffold was then inserted as an interposition graft using a 10-0 nylon suture for the end-to-end proximal and distal anastomoses. Finally, the intestines are returned to the abdominal cavity and the abdominal musculature and skin are closed in 2 layers with 5-0 absorbable suture. Aspirin (30 mg/L, Bayer) in their drinking water was administered to prevent excess graft thrombosis.

Blood Flow Measurements

Graft patency and blood flow was monitored with sonography at 2, 3, 8, 12, 16, 20, and 24 weeks post-implantation, which was then compared to blood flow from measurements taken prior to implantation of the grafts (baseline). Using color doppler, the abdominal aorta and grafts were visualized. In the distal abdominal aorta, before the iliac bifurcation, a pulse wave doppler spectrum was collected and analyzed for peak systolic velocity (PSV), end diastolic velocity (EDV), and mean velocity (MV) for the 3 largest waveforms for each mouse and timepoint. The resistivity index (RI) and pulsatility index (PI) were calculated as follows: RI=(PSV−EDV)/PSV and PI=(PSV−EDV)/MV.

Cryo-Sectioning

Samples were embedded in OCT compound (Electron Microscopy Sciences, Hatfield, Pa.) and frozen down on a dry ice/ethanol bath. Sections were cut at 50 μm using a Cryostat Microtom HM550 (Thermo Fisher Scientific) and collected on positive charged microscopy slides (Thermo Fisher Scientific).

Immunofluorescence Staining and Imaging

Samples were processed as previously described (Barreto-Ortiz, et al., 2015, Barreto-Ortiz, et al., 2013). Briefly, samples were fixed with 3.7% formaldehyde (Fisher Chemical, Fairlawn, N.J.) for 30 min, permeabilized with 0.1% Triton X-100 (Sigma-Aldrich) in PBS for 20 min, washed three times with PBS, and blocked overnight with 1% BSA. Samples were then incubated overnight at 4° C. with the indicated primary antibodies. Samples were rinsed three times with PBS before being incubated with the appropriate secondary antibodies or conjugated phalloidin at room temperature for 2 h. Samples were then rinsed with PBS three times, and counterstained with DAPI for 15 min. Images were obtained and processed using confocal microscopy (LSM 780, Carl Zeiss Inc., Thornwood, N.Y.). 2D epifluorescence images were obtained using an Olympus BX60 microscope.

Storage of Implantable Vascular Grafts

The fibrin hydrogel sdVGs were fabricated using the methods of the present invention. Briefly, fibrinogen is electrospun into a rotating thrombin bath to form aligned sheets of fibrin microfibers. Then, the aligned fibrin sheet is rolled both longitudinally and circumferentially around a Teflon-coated mandrel. The resulting multidirectional alignment of the fibers in the graft optimizes the amount of stress and strain the grafts can withstand before failure. Please see Regenerative and Durable Small-Diameter Graft as an Arterial Conduit. Proceedings of the National Academy of Sciences, 116 (26): 12710-12719 (2019); Elliott M B, Ginn B, Fukunishi T, et al. The grafts were then covalently crosslinked before a graded serial ethanol dehydration, removal from the mandrel, and storage (FIG. 4C, 9).

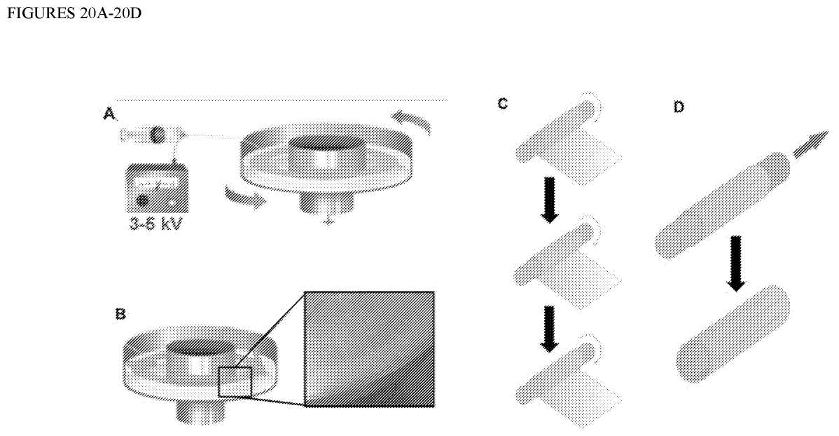

In another embodiment, fibrinogen was electrospun into a rotating thrombin bath to form aligned sheets of fibrin microfibers (FIG. 20A). Then, the aligned fibrin sheet was rolled both longitudinally and circumferentially around a Teflon-coated mandrel (FIGS. 20B-C). The resulting multidirectional alignment of the fibers in the graft optimizes the amount of stress and strain the grafts can withstand before failure. The grafts were then covalently crosslinked before a graded serial ethanol dehydration, removal from the mandrel, and storage (FIG. 20D).

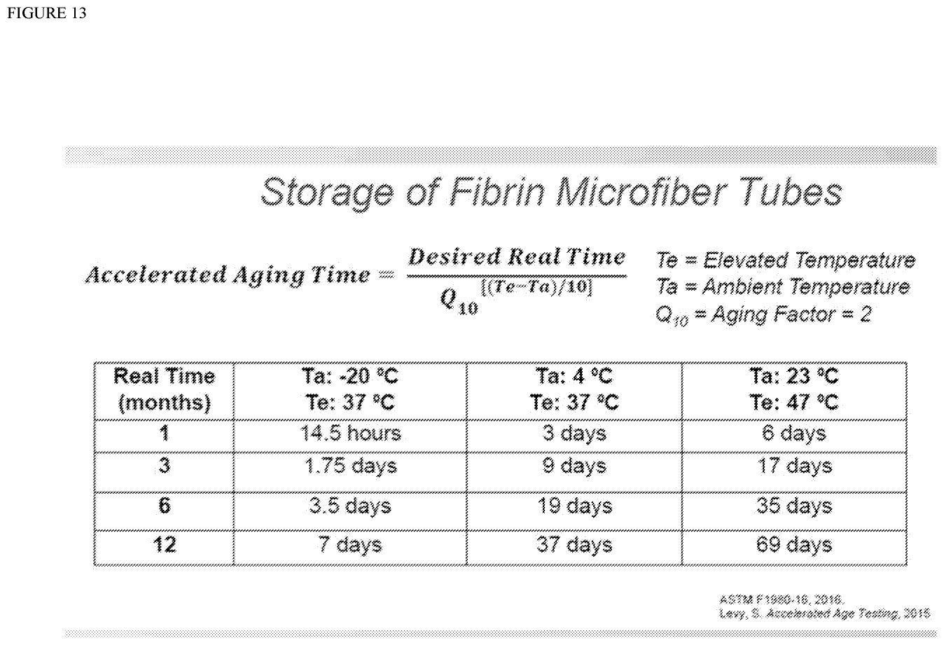

Storage of sdVGs: Dehydrated scaffolds were stored in parafilmed 6-well dishes to prevent moisture from rehydrating the scaffolds during long-term storage simulations in incubators with controlled humidity and temperature. Based on the ASTM International F1980-16 standards for accelerated aging, the inventors calculated an accelerated aging time at a specific elevated temperature for storage at a given ambient temperature and desired real time. As shown in FIG. 13, the inventors used a conservative aging factor of 2 to cover a range of potential packaging methods. To simulate ambient storage at −20° C. for a desired real time of 1, 3, 6, and 12 months, the grafts were stored in an elevated temperature of 37° C. for 0.60, 1.75, 3.5, and 7 days, respectively. To simulate ambient storage at 4° C. for a desired real time of 1, 3, 6, and 12 months, the grafts were stored in an elevated temperature of 37° C. for 3, 9, 19, and 37 days, respectively. To simulate ambient storage at 23° C. for a desired real time of 1, 3, 6, and 12 months, the grafts were stored in an elevated temperature of 47° C. for 6, 17, 35, and 69 days, respectively. Control grafts were left at 23° C. for no more than 5 days before rehydration and mechanical testing.

Mechanical Testing of sdVGs: To determine fibrin tube circumferential UTS and STF, the rehydrated sample was mounted radially onto an electromechanical puller (DMT560; Danish Myo Technology A/S, Aarhus, Denmark). After calibration and alignment, the pins were slowly moved apart using an electromotor at a rate of 50 μm/sec to apply radial stress on the specimen until breakage while displacement and force were recorded continuously. From these values, the stress, strain, Young's modulus, modulus of toughness, modulus of resilience, and plasticity were calculated.

The Examples/Methods have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter. The following Examples are offered by way of illustration and not by way of limitation.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### REFERENCES

ASTM International. ASTM F1980-16, 2016.
   Levy, S. D D L, Inc. Accelerated Age Testing, 2015.
Barreto-Ortiz S F, Fradkin J, Eoh J, et al. 2015, Fabrication of 3-dimensional multicellular microvascular structures, The FASEB Journal.
Barreto-Ortiz S F, Zhang S, Davenport M, et al. 2013, A novel in vitro model for microvasculature reveals regulation of circumferential ECM organization by curvature, PloS one, 8 (11): e81061.
Berger S, Jou L-D. 2000, Flows in stenotic vessels, Annual Review of Fluid Mechanics, 32 (1): 347-382.
Braverman I M, Yen A. 1977, Ultrastructure of the human dermal microcirculation, Journal of Investigative Dermatology, 68 (1): 44-52.
Brisbois E J, Davis R P, Jones A M, et al. 2015, Reduction in thrombosis and bacterial adhesion with 7 day implantation of S-nitroso-N-acetylpenicillamine (SNAP)-doped Elast-eon E2As catheters in sheep, J Mater Chem B, 3 (8): 1639-1645.
Bruce Alberts A J, Julian Lewis, Martin Raff, Keith Roberts, and Peter Walter. 2002, Molecular Biology of the Cell, Garland Science, New York.
Carmeliet P, Jain R K. 2000, Angiogenesis in cancer and other diseases, Nature, 407 (6801): 249-257.
Davis G E, Senger D R. 2005, Endothelial extracellular matrix biosynthesis, remodeling, and functions during vascular morphogenesis and neovessel stabilization, Circulation research, 97 (11): 1093-1107.
Elliott M B, Ginn B, Fukunishi T, et al. 2019, Regenerative and Durable Small-Diameter Graft as an Arterial Conduit, Proceedings of the National Academy of Sciences, 116 (26): 12710-12719.
Fleser P S, Nuthakki V K, Malinzak L E, et al. 2004, Nitric oxide-releasing biopolymers inhibit thrombus formation in a sheep model of arteriovenous bridge vascular grafts, Journal of vascular surgery, 40 (4): 803-811.
Fukunishi T, Best C A, Sugiura T, et al. 2016, Tissue-Engineered Small Diameter Arterial Vascular Grafts from Cell-Free Nanofiber PCL/Chitosan Scaffolds in a Sheep Model, PloS one, 11 (7): e0158555.
Gui, L., Muto, A., Chan, S. A., Breuer, C. K. & Niklason, L. E. Development of decellularized human umbilical arteries as small-diameter vascular grafts. Tissue Engineering Part A 15, 2665-2676 (2009).
Ives C, Eskin S, McIntire L. 1986, Mechanical effects on endothelial cell morphology: in vitro assessment, In vitro cellular & developmental biology, 22 (9): 500-507.
Jain R K. 2003, Molecular regulation of vessel maturation, Nature medicine, 9 (6): 685-693.
Jung S M, et al. (2013) Increased tissue transglutaminase activity contributes to central vascular stiffness in eNOS knockout mice. American Journal of Physiology-Heart and Circulatory Physiology 305(6):H803-H810.
Kelm J M, Lorber V, Snedeker J G, et al. 2010, A novel concept for scaffold-free vessel tissue engineering: self-assembly of microtissue building blocks, Journal of biotechnology, 148 (1): 46-55.
Khan O F, Chamberlain M D, Sefton M V. 2011, Toward an in vitro vasculature: differentiation of mesenchymal stromal cells within an endothelial cell-seeded modular construct in a microfluidic flow chamber, Tissue Engineering Part A, 18 (7-8): 744-756.
King K, Terai H, Wang C, et al. Microfluidics for tissue engineering microvasculature: endothelial cell culture. Micro Total Analysis Systems 2001: Springer; 2001. p. 247-249.
L'heureux N, Pâquet S, Labbé R, et al. 1998, A completely biological tissue-engineered human blood vessel, The FASEB Journal, 12 (1): 47-56.
Mozaffarian D B E, Go A S, Arnett D K, Blaha M J, Cushman M, de Ferranti S, Després J-P, Fullerton H J, Howard V J, Huffman M D, Judd S E, Kissela B M, Lackland D T, Lichtman J H, Lisabeth L D, Liu S, Mackey R H, Matchar D B, McGuire D K, Mohler E R 3rd, Moy C S, Muntner P, Mussolino M E, Nasir K, Neumar R W, Nichol G, Palaniappan L, Pandey D K, Reeves M J, Rodriguez C J, Sorlie P D, Stein J, Towfighi A, Turan T N, Virani S S, Willey J Z, Woo D, Yeh R W, Turner M B B; on behalf of the American Heart Association Statistics Committee and Stroke Statistics Subcommittee. 2015, Heart disease and stroke statistics—2015 update: a report from the American Heart Association, Circulation, 131 (4): e29-322.
Mulvany M J, Hansen O K, Aalkjaer C. 1978, Direct evidence that the greater contractility of resistance vessels in spontaneously hypertensive rats is associated with a narrowed lumen, a thickened media, and an increased number of smooth muscle cell layers, Circulation Research, 43 (6): 854-864.
Neumann T, Nicholson B S, Sanders J E. 2003, Tissue engineering of perfused microvessels, Microvascular Research, 66 (1): 59-67.
Niklason L E, Abbott W, Gao J, et al. 2001, Morphologic and mechanical characteristics of engineered bovine arteries, Journal of vascular surgery, 33 (3): 628-638.
Pashneh-Tala S, MacNeil S, Claeyssens F. 2015, The Tissue-Engineered Vascular Graft—Past, Present, and Future, Tissue Engineering Part B: Reviews, 22 (1): 68-100.
Quint C, Kondo Y, Manson R J, et al. 2011, Decellularized tissue-engineered blood vessel as an arterial conduit, Proceedings of the National Academy of Sciences, 108 (22): 9214-9219.
Robinson, J. Value-Based Purchasing For Medical Devices. Health Affairs 27, 1523-1531, doi:10.1377/hlthaff.27.6.1523 (2008).
Schriefl A J, Zeindlinger G, Pierce D M, et al. 2011, Determination of the layer-specific distributed collagen fibre orientations in human thoracic and abdominal aortas and common iliac arteries.
Steppan J, et al. (2014) Exercise, vascular stiffness, and tissue transglutaminase. Journal of the American Heart Association 3(2):e000599.
Sundaram, S., Echter, A., Sivarapatna, A., Qiu, C. & Niklason, L. Small-diameter vascular graft engineered using human embryonic stem cell-derived mesenchymal cells. Tissue Engineering Part A 20, 740-750 (2014).
Syedain Z, Reimer J, Lahti M, et al. 2016, Tissue engineering of acellular vascular grafts capable of somatic growth in young lambs, Nature Communications, 7.
Thompson, C. A. et al. A novel pulsatile, laminar flow bioreactor for the development of tissue-engineered vascular structures. Tissue engineering 8, 1083-1088 (2002).
van Hinsbergh V W. Endothelium—role in regulation of coagulation and inflammation. Seminars in immunopathology: Springer; 2012. p. 93-106.
Williams B. 1998, Mechanical influences on vascular smooth muscle cell function, Journal of hypertension, 16 (12): 1921-1929.
Williams C, Wick T M. 2004, Perfusion bioreactor for small diameter tissue-engineered arteries, Tissue engineering, 10 (5-6): 930-941.
Zhang S, Liu X, Barreto-Ortiz S F, et al. 2014, Creating polymer hydrogel microfibres with internal alignment via electrical and mechanical stretching, Biomaterials, 35 (10): 3243-3251.
Zhang S, Liu X, Barreto Ortiz S, et al. Creating Polymer Hydrogel Microfibers with Internal Alignment via Electrical and Mechanical Stretching. 2013.
Zheng Y, Chen J, Craven M, et al. 2012, In vitro microvessels for the study of angiogenesis and thrombosis, Proceedings of the National Academy of Sciences, 109 (24): 9342-9347.

1. A tubular scaffold comprising a hollow core surrounded by one or more sheets comprising dehydrated or hydrated hydrogel nanofibers having internally aligned polymer chains.

2. The tubular scaffold of claim 1 wherein at least one of the sheets comprises longitudinally aligned dehydrated or hydrated hydrogel nanofibers.

3. The tubular scaffold of claim 1 wherein at least one of the sheets comprises circumferentially or otherwise angled relative to the tubular scaffold longitudinal axis aligned dehydrated or hydrated hydrogel nanofibers.

4. The tubular scaffold of claim 1 wherein the hollow core has an inner diameter in the range of 0.1 mm to 6 mm;

5. The tubular scaffold of claim 1 wherein the one or more sheets have a combined thickness in the range of 5 nm to 2000 μm.

6. The tubular scaffold of claim 1 having an average circumferential Ultimate Tensile Stress (UTS) in a range of 40 kPa to 1400 kPa and an average circumferential Strain to Failure (STF) in a range of 1 to 13.

7. The tubular scaffold of claim 1 having an elastic modulus in the range of 20 to 300 kPa.

8. The tubular scaffold of claim 1 having a toughness in the range of 40 kPa to 1000 kPa.

9. The tubular scaffold of claim 1 having a modulus of resilience in the range of 15 kPa to 1000 kPa.

10. The tubular scaffold of claim 1, wherein said scaffold is capable of being stored in a dehydrated state for up to one year at −20 to 23 degrees C.

11. A cellularized vascular graft comprising:
a tubular scaffold comprising a hollow core surrounded by one or more sheets comprising hydrated hydrogel nanofibers with internal polymer alignment; and
one or more cell layers attached to the tubular scaffold.

12. The cellularized vascular graft of claim 11 wherein at least one of the sheets comprises longitudinally aligned hydrated hydrogel nanofibers.

13. The cellularized vascular graft of claim 11 wherein at least one of the sheets comprises circumferentially or otherwise angled relative to the tubular scaffold longitudinal axis aligned hydrated hydrogel nanofibers.

14. The cellularized vascular graft of claim 11 wherein the hollow core has an inner diameter in the range of 0.1 mm to 6 mm.

15. The cellularized vascular graft of claim 11 wherein the one or more sheets have a combined thickness in the range of 5 nm to 2000 μm.

16. The cellularized vascular graft of claim 11 wherein the one or more cell layers have a combined thickness in the range of 10 μm to 300 μm.

17. The cellularized vascular graft of claim 11 wherein the cells comprise endothelial colony forming cells that align in the direction of flow on the tubular scaffold.

18. A method for treating vascular damage comprising:
administering a vascular graft of claim 11 to a subject with vascular damage; and
treating the vascular damage of the subject.

19. The method of claim 18 wherein the vascular graft is administered by vascular bypass surgery.

20. The method of claim 18 wherein the vascular damage is to an artery or vein.

21. The method of claim 18 wherein the vascular damage is caused by a disease or trauma.

22. The method of claim 18, wherein the vascular damage is selected from the group consisting of congenital cardiovascular defect (CCD), coronary artery disease (CAD), or peripheral artery disease (PAD).

23. A mesh comprising sheets comprising dehydrated or hydrated hydrogel nanofibers having internally aligned polymer chains wherein each sheet has a controlled nanofiber orientation that is longitudinal, perpendicular, or otherwise angled.

24. The mesh of claim 23, wherein the one or more sheets have a combined thickness in the range of 5 nm to 2000 μm.

25. The mesh of claim 23, wherein said mesh has an average circumferential Ultimate Tensile Stress (UTS) in a range of 40 kPa to 1400 kPa and an average circumferential Strain to Failure (STF) in a range of 1 to 13.

26. The mesh of claim 23, wherein said mesh has an elastic modulus in the range of 20 to 300 kPa.

27. The mesh of claim 23, wherein said mesh has in the range of 40 kPa to 1000 kPa.

28. The mesh of claim 23, wherein said scaffold is capable of being stored in a dehydrated state for up to one year at −20 to 23 degrees C.

29-31. (canceled)
